(19) United States
(12) Patent Application Publication
(10) Pub. No.: US 2021/0361771 A1
(43) Pub. Date: Nov. 25, 2021

(54) TERPENE COUPLING CONJUGATE

(71) Applicant: LABORATOIRES ERIGER, CHAMBRAY LES TOURS (FR)

(72) Inventors: Eric BUCHY, BALLAN-MIRE (FR); Emilie MUNNIER, TOURS (FR); Igor CHOURPA, SAVONNIERES (FR)

(21) Appl. No.: 17/324,615

(22) Filed: May 19, 2021

(30) Foreign Application Priority Data
May 20, 2020 (FR) 2005150

(51) Int. Cl. A61K 47/54; A61K 31/192; A61K 31/167; A61K 31/522; A61K 31/07; A61K 31/455; A61K 31/375; A61K 31/355; A61K 31/122; A61K 31/05; A61K 38/44

(52) U.S. Cl. CPC A61K 47/54; A61K 31/192; A61K 31/167; A61K 31/522; A61K 31/07; A61K 38/446; A61K 31/375; A61K 31/355; A61K 31/122; A61K 31/05; A61K 31/455

(57) ABSTRACT

The subject matter of the present invention relates to the use of an optionally branched linear terpene having at most one C═C unsaturation for the production of conjugates provided with self-assembly properties, as well as a self-assembly agent of formula (I):

X(-Spacer-Y-Terpene)ₚ (I)

in which:
“Terpene” is linear, optionally branched, having at most one C═C unsaturation;
“Y” is a bond or a molecular fragment with a biodegradable bond;
“Spacer” is a bond or a fragment comprising at least one carbon atom;
“X” is a molecular fragment comprising at least one biodegradable bond;
“p” ranges between 0.1 and 4; and
the “-Spacer-Y-” group optionally can be a bond,
   as well as the conjugate resulting from the combination of the self-assembly agent of formula (I) with an active molecule MA.

### CROSS-REFERENCE TO RELATED PATENT APPLICATIONS

This application claims the benefit of priority to French Patent Application No. 2005150 filed May 20, 2020, which is incorporated by reference in its entirety.

### FIELD OF THE INVENTION

The present invention relates to a coupling conjugate via a biodegradable bond between a particular terpene and a molecule of interest making it possible to obtain nanoparticles for, for example, the administration of active molecules.

### TECHNOLOGICAL BACKGROUND

The class of terpenes, as is well known in chemistry, is known for the formation of nanoparticles that also can be coupled with molecules of interest, such as molecules for pharmaceutical purposes, to improve, or even allow, their bioavailability.

For example, WO2015/173367 discloses a conjugate of oxazaphosphorine and of geranyl of formula (I), as described in this document, which can self-assemble into nanoparticles.

WO2014/091436 discloses nanoparticles comprising at least one macromolecule of the glycosaminoglycan type (such as fondaparinux or derivative) non-covalently coupled to at least one cationic hydrocarbon molecule with a squalene nature.

FR2988092 discloses a complex of 5-(1,2-dihydroxy-ethyl)-3,4-dihydroxy-5H-furan-2-one (vitamin C) or derivative covalently linked to at least one hydrocarbon radical, such as squalene, farnesol, geraniol, etc. of formula (A), as described in this document. FR2988092 particularly discloses that the products self-assemble into nanoparticles in the aqueous phase.

WO2010/049899 relates to a complex formed from at least one beta-lactam molecule covalently coupled to at least one hydrocarbon radical comprising 18 carbon atoms and containing at least one 2-methyl-buta-2-ene unit (more specifically with a squalene nature), nanoparticles of these complexes and their preparation method. It can be seen (for example, in claim 1 of this document) that the complex comprises at least one statin.

WO2010/049900 relates to a complex formed from at least one statin molecule covalently coupled to at least one hydrocarbon radical comprising 18 carbon atoms and containing at least one 2-methyl-buta-2-ene unit (more specifically with a squalene nature), nanoparticles of these complexes and their preparation method. It can be seen (for example, in claim 1 of this document) that the complex comprises at least 3 double bonds.

WO2009/150344 relates to a complex formed from at least one nucleic acid molecule comprising between 10 and 40 nucleotides, covalently coupled to at least one hydrocarbon compound, which is at least one C18 hydrocarbon compound, having a squalene structure or a structure similar thereto.

WO2009/071850 relates to a water-dispersible derivative of a therapeutic agent having low solubility in water, which comprises at least one molecule of said agent covalently linked to at least one molecule of a hydrocarbon derivative having a squalene or similar structure.

FR2874016 relates to nanoparticles of derivatives of gemcitabine, more particularly 2,2′-difluoro-2′-deoxycytidine derivatives of formula (I), as described in this document. The substituent groups in this formula I can be C18 hydrocarbon-based acyl radicals, and more particularly squalenoyl radicals. The function of squalenoyl is also provided in this document: to retain its ability to compact or to cause a significant reduction in surface tension or even a rapid drop in surface tension when it is placed in the presence of a polar solvent.

FR 2608988 and FR2608942 relate to the preparation of dispersible colloidal systems of substances in the form of nanoparticles.

Thus, all the cited prior art relates to nanoparticles comprising terpenes with several double bonds, conferring their ability to compact or even to cause a significant reduction in surface tension or even a rapid drop in surface tension when they are placed in the presence of a polar solvent. Enrichment in unsaturated bonds (e.g. polarizable) allows this effect. However, the Applicant has surprisingly discovered that terpenes having a much lower level of unsaturation also can be used. This introduces interesting perspectives in terms of the supply of products, which particularly can be bio-sourced.

Thus, and more specifically, the present invention relates to a formed conjugate capable of spontaneously self-assembling in water into nanoobjects having a size ranging from a few tens to a few hundred nanometers, which allows the molecule of pharmaceutical, veterinary, phytosanitary or cosmetic interest to be protected against early biodegradation. The degradation, in a biological medium, of the bond between the phytol (or other terpene) allows the molecule of interest to be released. The invention therefore allows an improvement in the bioavailability and/or the pharmacokinetic characteristics of the molecule of interest.

### SUMMARY OF THE INVENTION

The subject matter of the present invention therefore relates to the use of an optionally branched linear terpene having at most one C═C unsaturation for the production of conjugates provided with self-assembly properties.

In addition, the subject matter of the present invention relates to a self-assembly agent of formula (I):

[Chem. 1]

X(-Spacer-Y-Terpene)ₚ (I)

in which:
“Terpene” is as defined herein, i.e. it can be an optionally branched linear terpene having at most one C═C unsaturation;
“Y” is a bond or a molecular fragment with a biodegradable bond;
“Spacer” is a bond or a fragment comprising at least one carbon atom;
“X” is a molecular fragment comprising at least one biodegradable bond;
“p” ranges between 0.1 and 4; preferably p is an integer equal to 1 or 2; and
the “-Spacer-Y-” group optionally can be a bond.

In addition, the subject matter of the present invention relates to a conjugate provided with self-assembly properties of formula (II):

[Chem. 2]

MA(-AA)ₖ (II)

in which:
“AA” is a self-assembly agent as defined herein;
“MA” is a biologically active molecule; and
“k” ranges between 0.1 and 6; preferably k is an integer equal to 1 or 2;
as well as to these pharmaceutically acceptable salts and/or solvates.

The subject matter of the present invention also relates to a method for self-assembly in an aqueous medium, wherein a conjugate according to formula (II) above (1) in solution in a water-miscible solvent S1, is (2) nanoprecipitated in water, then (3) the solvent S1 at least is evaporated under reduced pressure.

The subject matter of the present invention also relates to a pharmaceutical, veterinary and/or cosmetic formulation comprising a self-assembly agent of formula (I), as described above.

The subject matter of the present invention also relates to a pharmaceutical, veterinary and/or cosmetic formulation comprising a self-assembly conjugate of formula (II), as described above.

### Definitions

Within the scope of the present invention, the term “optionally branched linear terpene” is understood to mean a hydrocarbon, the number of carbons of which is a multiple of five, comprising a linear chain of carbons, optionally branched by C1 to C4 alkyl groups. The C1-C4 alkyl groups comprise methyl, ethyl, propyl and butyl groups, preferably the methyl and ethyl groups.

Within the scope of the present invention, the term “unsaturation” is understood to mean a double bond between two atoms, such as two carbon atoms in the case of an alkene, for example.

Within the scope of the present invention, the term “self-assembly” is understood to mean that the molecules spontaneously assemble into particles when said particles are stimulated or conditioned to this end (for example, in the presence of water). Depending on the size of the particles thus formed, this will involve nanoparticles (the sizes of which are of the order of one nanometer to one or two hundred nanometers), or microparticles (the sizes of which are of the order of one micrometer to approximately five hundred micrometers).

Within the scope of the present invention, the term “self-assembly agent” is understood to mean an agent, i.e. a molecular fragment allowing self-assembly as defined above.

Within the scope of the present invention, the term “biodegradable bond” is understood to mean a chemical bond (covalent or electrostatic, e.g. ionic) that can be broken by a biological means, i.e. resulting from a biological system, for example, an enzyme or an acid. Thus, breaking the bond can involve at least one water molecule; it then will be a matter of hydrolysis.

Within the scope of the present invention, the term “biologically active molecule” is understood to mean any molecule having a biological effect, which can have a more general physiological effect on the considered biological entity. A “biological effect” can be identified by a comparison between at least one treated biological entity and at least one identical or similar biological entity without treatment.

Within the scope of the present invention, the term “nanoprecipitation” is understood to mean a self-assembly of molecules, such as that which has been defined above, causing the formation and the separation of the liquid in which it was dissolved in the form of one or more nanometric sized particle(s).

Within the scope of the present invention, the term “pharmaceutically acceptable” refers to compositions, compounds, salts and the like that are, according to sound medical judgment, adapted for contact with the tissues of the subject, or which can be administered to the subject without excessive toxicity or other complications commensurate with a reasonable benefit/risk ratio. Thus, the term “pharmaceutically acceptable salt” can refer to non-toxic salts, which generally can be prepared by bringing the compound of the invention into contact with an appropriate organic or inorganic acid. For example, pharmaceutical salts can be, but are not limited to, acetates, benzenesulfonates, benzoates, bicarbonates, bisulfates, bitartrates, bromides, butyrates, carbonates, chlorides, citrates, diphosphates, fumarates, iodides, lactates, laurates, malates, maleates, mandelates, mesylates, oleates, oxalates, palmitates, phosphates, propionates, succinates, sulphates, tartrates and similar compounds.

Within the scope of the present invention, the term “solvate” or the term “pharmaceutically acceptable solvate” refers to a solvate formed from the association of one or more molecules of compounds of the invention with one or more molecules of solvent. The term solvates comprises hydrates, such as hemihydrate, monohydrate, dihydrate, trihydrate, tetrahydrate and the like.

### DETAILED DESCRIPTION

Use

The subject matter of the present invention therefore relates to the use of an optionally branched linear terpene having at most one C═C unsaturation for the production of conjugates provided with self-assembly properties.

The subject matter of the present invention relates to the use as described herein, characterized in that the terpene comprises between 15 and 25 carbon atoms.

The subject matter of the present invention more particularly relates to the use as described herein, characterized in that the terpene is bio-sourced.

The term “bio-sourced” is understood, within the scope of the present invention, to mean that the compounds that can be provided in a few steps (extraction, treatment with an acid, treatment with a base, precipitation, etc.) originate from the biomass. By contrast, an organic synthesis product is produced from chemical and/or petrochemical products.

Preferably, the present invention relates to the use as described herein, characterized in that the terpene is phytol or a derivative of phytol, such as isophytol.

Phytol has the following formula:

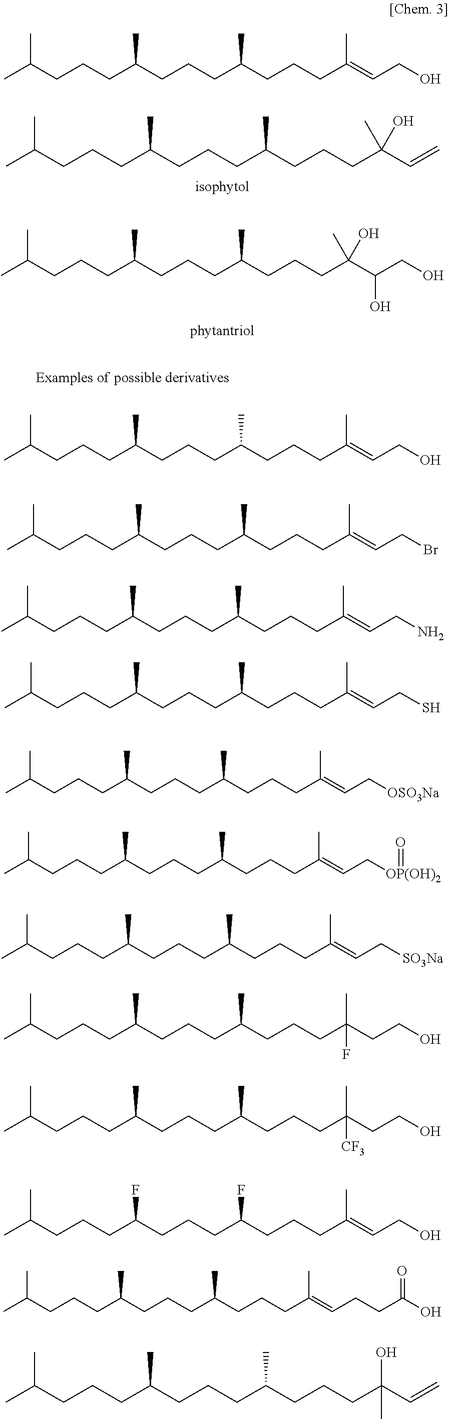

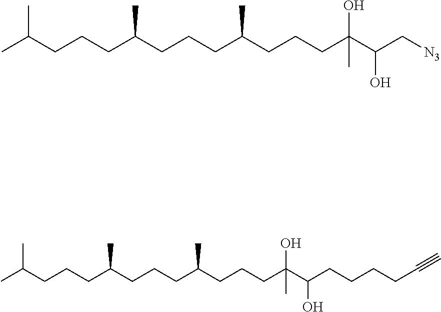 [SMILES: CC(C)CCC[C@@H](C)CCC[C@@H](C)CCCC(C)(O)C(O)CN=[N+]=[N-] ; C#CCCCCC(O)C(C)(O)CCC[C@H](C)CCC[C@H](C)CCCC(C)C]

The term “derivative” can denote, within the scope of the present invention, an isomer of the relevant product. For example, a phytol derivative can be isophytol, or even phytantriol. A derivative can also relate to the relevant product with a grafted substituent selected from a halogen, —OH, —NH₂, —CH₃, —C(O)OH, or even —C(O)OR, where R independently is an alkyl in C1-C4.

Self-Assembly Agent

The subject matter of the present invention relates to the self-assembly agent of formula (I) as described above.

In one embodiment, the spacer can be a C1-C10 hydrocarbon chain optionally substituted with one or more substituents selected from —OH, C1-C4 alkyl and C1-C4 alkyloxy, optionally comprising:
one or more heteroatoms, such as S, N and 0;
one or more chemical groups, such as —NHC(O)—, —OC(O)—, OC(O)O, —NH—, —NHC(O)—NH—, —SS— and —CR═N—NH—C(O)—, —ONH—, —ONR—, —O—C(═S)—S—, —C(═S)—S—, where R is independently H, an aryl group, or an alkyl group, such as a C1-C6 alkyl group, preferably a C1-C3 alkyl group;
one or more heteroaryl or aryl groups; and/or
one or more aliphatic rings or heterocyclic rings, preferably comprising from 4 to 6 atoms, and optionally substituted by one or more substituents selected from the —OH, C1-C4 alkyl and C1-C4 alkyloxy groups.

Within the scope of the present invention, an “aryl” group refers to an unsubstituted or substituted aromatic ring. Preferably, the aryl group is a phenyl group optionally substituted by one or more groups, such as C1-C4 alkyl, C1-C4 alkyloxy, OH or halogen atoms.

Within the scope of the present invention, a “heteroaryl” refers to an aromatic ring system, in which one or more aromatic atoms is/are a heteroatom, such as N, O or S. The heteroaryl group can be substituted or unsubstituted and preferably comprises from 4 to 6 ring atoms. Examples of heteroaryl groups are, but are not limited to, pyridinyl, pyridazinyl, pyrimidyl, pyrazyl triazinyl, pyrrolyl, pyrazolyl, imidazolyl, triazolyl, pyrazinyl, pyrimidinyl, tetrazolyl, furyl, thienyl, isoxazolyl, thiazolyl or oxazolyl.

Within the scope of the present invention, an “aliphatic heterocyclic ring” refers to a non-aromatic ring system, in which one or more aromatic atoms is/are a heteroatom, such as N, O or S. The heteroaryl group can be substituted or unsubstituted and preferably comprises from 4 to 6 ring atoms. Examples of aliphatic heterocycles are, but are not limited to, morpholine, piperazine, pyrrolidine, dioxane, piperidine, tetrahydrofuran and similar fragments.

In one embodiment, the spacer can comprise a polyether group, such as polyethylene glycol or polypropylene glycol, preferably comprising from 2 to 6 monomers.

In one embodiment, the spacer can be selected from the group made up of:
amino acids and the derivatives thereof;
peptides comprising from 2 to 10, preferably from 2 to 5 amino acids and the derivatives thereof;
C1-C10 hydrocarbon chains optionally linked to one or more heteroatoms, such as S, N and/or O, and/or to one or more chemical groups, such as —NHC(O)—, —OC(O)—, —NH—, —NH—C(O)—NH—, —SS— and —CH═N—NH—C(O)— and/or one or more heteroaryl or aryl groups, said hydrocarbon chains optionally being substituted by one or more substituents selected from —OH, C1-C4 alkyl and C1-C4 alkoxy groups; and
the combinations thereof.

In one embodiment, the spacer is selected from amino acids, dipeptides and the derivatives thereof. For example, the spacer can be based on citrulline, lysine, ornithine, alanine, phenylalanine, cysteine, glycine, valine, leucine and the dipeptides thereof.

In another embodiment, the spacer can be selected from the following fragments: —NH—, —O—, —S—, —NR—, —ONH—, —ONR—, —OC(O)O—, —OC(S)S—, —N(R)C(S)S—, and the combinations thereof, where R independently is an alkyl, preferably a C1-C3 alkyl, optionally with polyether groups, such as polyethylene glycol or polypropylene glycol, preferably comprising from 2 to 6 monomers on one side or the other of said fragments.

In another embodiment, the spacer can be Y1-(CH2)m-Y2, with m being an integer from 1 to 8 or Y1-(CH2-CH2-O)q-CH2-CH2-Y2, with q being an integer from 1 to 5, where Y1 and Y2 are independently selected from —O—, —NH—, —S—, —OC(O)—, —C(O)NR—, —C(O)NH—, —NHC(O)—, —OC(S)—S—, —NR—, —ONH—, —ONR—, —OC(O)—O—, NRC(S)S— and —C(O)O—, with R independently being an alkyl, preferably a C1-C3 alkyl. In a particular embodiment, the spacer can be Y1-(CH2)m-Y2, where m is an integer from 1 to 6, preferably from 1 to 4 and Y1 and Y2 are independently selected from —O—, —NH—, —S—, —C(O)NH—, —NHC(O)—, —OC(O)— and —C(O)O—.

In addition, in the self-assembly agent of formula (I) as described above, p advantageously can range between 0.5 and 3.5, between 0.7 and 3, or between 0.9 and 2.5. Preferably, p is a substantially whole number selected from 1, 2, 3 and 4. The term “substantially” is understood herein to be a variation of plus or minus 0.1.

In one embodiment, the present invention relates to a self-assembly agent of formula (I) as described above, characterized in that the spacer comprises, or is made up of, any one of the following fragments:

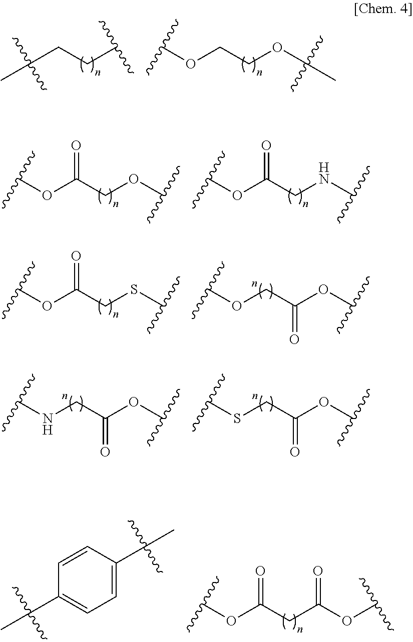

in which “n” are independently whole numbers ranging between 0 and 6, preferably ranging between 1 and 4.

In one embodiment, the present invention relates to a self-assembly agent of formula (I) as described above, characterized in that said at least one biodegradable bond of “X” comprises an ionic bond and/or the biodegradable bond of “Y” is a covalent bond.

In one embodiment, the present invention relates to a self-assembly agent of formula (I) as described above, characterized in that “Y” and/or “X” comprise, or are made up of, any one of the following fragments:
fragments comprising one or more heteroatoms, such as S, N and O, and/or one or more chemical groups, such as —NH—, —O—, —S—, —NR—, —ONH—, —ONR—, —NHC(O)—, —OC(O)O—, —OC(O)—, —NH—C(O)—NH—, —OC(S)S—, —N(R)C(S)S—, —SS—, —CH═N—NH—C(O)— and the combinations thereof, where R independently is an alkyl (preferably a C1-C3 alkyl) or a heteroaryl or aryl group;
C1-C10 hydrocarbon chains linked to one or more heteroatoms, such as S, N and/or O, and/or one or more chemical groups, such as —NHC(O)—, —OC(O)—, —NH—, —NH—C(O)—NH—, —SS—, —CH═N—NH—C(O)—, heteroaryl or aryl, said hydrocarbon chains optionally being substituted by one or more substituents selected from —OH, C1-C4 alkyl and C1-C4 alkoxy groups; and
the combinations thereof.

In one embodiment, the present invention relates to a self-assembly agent of formula (I) as described above, characterized in that “Y” and/or “X” comprise, or are made up of, any one of the following fragments: —NH—, —O—, —S—, —NR—, —ONH—, —ONR—, —OC(O)O—, —OC(S)S—, —N(R)C(S)S—, —C(O)O—, —C(O)NH—, —NHC(O)NH—, —N═C—, —SS—, and the combinations thereof, where R independently is an alkyl, preferably a C1-C3 alkyl, optionally with polyether groups, such as polyethylene glycol or polypropylene glycol, preferably comprising from 2 to 6 monomers on one side or the other of said fragments.

In one embodiment, the present invention relates to a self-assembly agent of formula (I) as described above, characterized in that “Y” and/or “X” comprise at least one ionic fragment, for example, —NH₃⁺, —CO₂⁻, —PO₄⁻, —SO₃⁻, —SO₄²⁻ and/or —NR₃⁺, where R independently is a C1-C4 alkyl.

In one embodiment, the present invention relates to a self-assembly agent of formula (I) as described above, characterized in that “Y” and/or “X” comprise at least one trivalent fragment, such as —C(—O—)2, —B(—O—)2, and/or —O—PO(—O—)2.

The term “trivalent” is understood, within the scope of the present invention, to mean that the fragment is able to bond to three other functions. The active molecule can comprise one or more binding functions. Thus, when “Y” and/or “X” comprise at least one trivalent fragment, such as —C(—O—)2, —B(—O—)2, and/or —O—PO(—O—)2, the ratio between the fragment “Y” (and/or “X”), and MA can be 1:1 and/or 1:2. For example, two fragments of active molecules “MA” and a fragment “-Spacer-Y-Terpene,” or two fragments “-Spacer-Y-Terpene” and a fragment of active molecule “MA.” Preferably, the fragments with 2 bonds represent acetals and boron acetals and these functions describe a bond to the same molecule, that is a 1:1 complex between the terpene (i.e. the fragment of formula (I) comprising Y and/or X) and MA.

In one embodiment, the present invention relates to a self-assembly agent of formula (I) as described above, characterized in that “Y” and/or “X” comprise, or are made up of, any one of the following fragments:

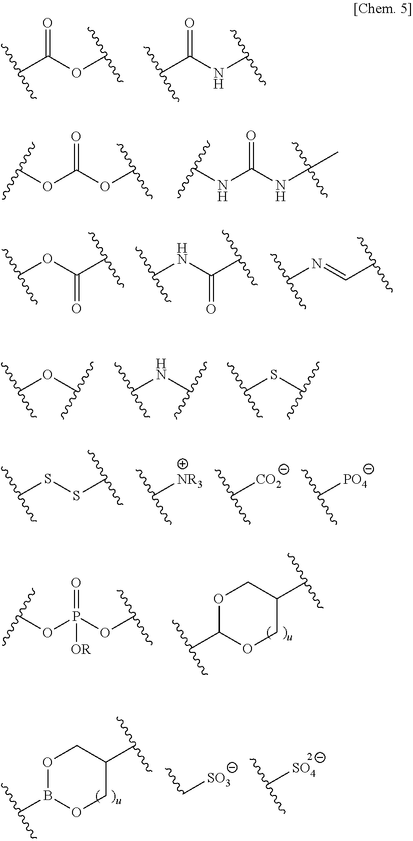

in which:
“u” are independently whole numbers ranging between 0 and 6, preferably between 0 and 1.
“R” is a hydrogen atom, a C1-C6 alkyl group, a C4-C8 aromatic group or a monocyclic or polycyclic (C1-C6)-aryl (C4-C8) alkyl group; for example, R can represent a hydrogen atom, a methyl, ethyl, propyl, butyl, phenyl, or even a benzyl group.

Conjugate Provided with Self-Assembly Properties of Formula (II)

The subject matter of the present invention relates to the conjugate provided with self-assembly properties of formula (II) as described above.

The bond between MA and AA in the conjugate provided with self-assembly properties of formula (II) can be covalent (referred to herein as “covalent form”) or ionic (referred to herein as “ionic form”).

The subject matter of the present invention thus can relate to a conjugate provided with self-assembly properties of formula (II) comprising an active ingredient MA, such as, for example, a drug, known to have low bioavailability, such as paclitaxel.

Examples of an active pharmaceutical ingredient (MA) include antimicrobial agents, anti-acne agents, anti-inflammatory agents, analgesic agents, anesthetic agents, antihistamine agents, antiseptic agents, immunosuppressants, antihemorrhagic agents, vasodilators, wound healing agents, anti-biofilm agents and the mixtures thereof.

In addition, the subject matter of the present invention thus can relate to a conjugate provided with self-assembly properties of formula (II) comprising an active ingredient MA, such as, for example, a cosmetic ingredient.

Examples of cosmetic ingredients (MA) include 4-nBu-resorcinol, 6-nHex-resorcinol, caffeic acid, ferulic acid, kojic acid, biotin, adenosine mono-phosphate, adenosine tri-phosphate, aescin, arbutin, bakuchiol, bisabolole, boldine, caffeine, canabidiol, coenzyme A, coenzyme Q10, dihydroxy acetone, D-panthenol, glabridin, idebenone, L-carnitine, licochalchone A, N-acetyl-tetrapeptide-2, N-acetyl-tetrapeptide-9, niaccinamide, oleuropein, resorcinol, resveratrol, tripeptide-29, vanillin, vitamin A, vitamin B3, vitamin B8, vitamin C and vitamin E.

In addition, the subject matter of the present invention thus can relate to a conjugate provided with self-assembly properties of formula (II) comprising an active ingredient MA, such as, for example, a phytosanitary ingredient.

Examples of phytosanitary ingredients (MA) include: benzoic acid, benalaxyl, bromoxynil, captan, carbendazim, carfentrazone, carvone, daminozide, dicamba, difenoconazole, epoxiconazole, fenhexamide, flazasulfuron, fludioxonyl, glyphosate, isoproturon, iprodione, imidacloprid, imazalil, MCPA, mecoprop, etconazol, propiconazole, sulfosulfuron, warfarin and peptides of structures YDPAPPPPPP, TDVDHVFLRF-amide, SDVDHVFLRF-amide.

The following particularly can be cited as an active molecule MA: amlodipine, gallopamil, verapamil, barnidipine, felodipine, isradipine, lacidipine, verapamil, quinidine, amiodarone, reversin, matairesinol, sipholenol and cyclosporine, lercanidipine, nicardipine, nifedipine, nimodipine, nisoldipine, nitrendipine or diltiazem.

In one embodiment, the present invention relates to a conjugate of formula (II) as described above, characterized in that MA is selected from ibuprofen, paracetamol, 4-nBu-resorcinol, 6-nHex-resorcinol, azelaic acid, caffeic acid, ferulic acid, glycyrrhizic acid, hyaluronic acid, kojic acid, linoleic acid, lipoic acid, biotin, di-phosphate, adenosine mono-phosphate, adenosine tri-phosphate, aescin, arbutin, bakuchiol, bis-(Et)-Hexyl-dihydroxymethoxybenzyl-malonate, bisabolole, boldine, caffeine, canabidiol, coenzyme A, coenzyme Q10, dihydroxy acetone, dihydroxymethylchromonyl-palmitate, D-panthenol, ectoin, glabridin, idebenone, L-carnitine, licochalchone A, menthol, N-acetyl-tetrapeptide-2, N-acetyl-tetrapeptide-9, niaccinamide, oleuropein, phycocyanin, pro-xylan, resorcinol, resveratrol, tripeptide-29, tyamine pyrrophosphate, vanillin, vitamin A, vitamin B3, vitamin B8, vitamin C and vitamin E.

In one embodiment, the present invention relates to a conjugate of formula (II) as described above, characterized in that MA is selected from the following active ingredients: methylprednisolone, dexamethasone, cortisone, ibuprofen, naproxen, flurbiprofen, ketoprofen, vitamin C, carnosic acid, astaxanthin, vitamin B1, vitamin B6, vitamin B12, β-carotene derivatives, lutein, allantoin, vitamin A, folic acid, vancomycin, rifampicin, quaternary ammonium salts and chlorhexidine.

In one embodiment, the present invention relates to a conjugate of formula (II) as described above, characterized in that the size of MA is less than 20 kDa, preferably less than 15 kDa, more preferably less than 10 kDa, even more preferably less than 5 kDa, such as less than 3 kDa or less than 2 kDa.

In one embodiment, the present invention relates to a conjugate of formula (II) as described above, characterized in that MA is an antimicrobial agent selected from the following active ingredients: penicillins and related drugs, carbapenems, cephalosporins aminoglycosides and related drugs, erythromycin, bacitracin, mupirocin, chloramphenicol, thiamphenicol, sodium fusidate, lincomycin, clindamycin, macrolides, novobiocin, vancomycogramin, streptogramins, anti-folate agents including sulfonamides, trimethoprim and the combinations thereof and pyrimethamine, synthetic antibacterials including nitrofurans, methazolamide, mandelate and hippurate methazolamide, nitroimidazoles, quinolones, fluoroquinolones, isoniazid, ethambutol, pyrazinamide, para-aminamide (PAS), cycloserine, capreomycin, prothionamide, thiacetazone, viomycin, spiramycin, glycopeptide, a glycylcycline, ketolides, oxazolidinone, imipenene, amikacin, netilmicin, fosfomycin, gentamycin, ceftriaxone, aztreonam and metronidazole, epiroprim, sanfetrinem sodium, biapenem, dynemicin, cefluprenam, cefoselifin, sulopenem, cyclothialidine, carumonam, cefozopran and cefetamet pivoxil.

In one embodiment, the present invention relates to a conjugate of formula (II) as described above, characterized in that MA is a topical anti-acne agent selected from the following active ingredients: adapalene, azelaic acid, clindamycin (for example, clindamycin phosphate), doxycycline (for example, doxycycline monohydrate), erythromycin, keratolytics, such as salicylic acid and retinoic acid (Retin-A″), norgestimate, organic peroxides, retinoids, such as isotretinoin and tretinoin, sodium sulfacetamide, tazarotene and acetretin.

In one embodiment, the present invention relates to a conjugate of formula (II) as described above, characterized in that MA is an antihistamine agent selected from the following active ingredients: diphenhydramine hydrochloride, diphenhydramine salicylate, diphenhydramine, chlorpheniramine hydrochloride, isothipendyl chlorpheniramine maleate hydrochloride, tripelennamine hydrochloride, promethazine hydrochloride, methdilazine hydrochloride and the like. Examples of local anesthetic agents that can be used as the “MA” group in the conjugate of formula (II) as described above include dibucaine hydrochloride, dibucaine, lidocaine hydrochloride, lidocaine, benzocaine, p-Butylaminobenzoic acid 2-(di-ethylamino) ethyl ester hydrochloride, procaine hydrochloride, tetracaine, tetracaine hydrochloride, chloroprocaine hydrochloride, oxyprocaine hydrochloride, mepivacaine, cocaine hydrochloride, piperocaine hydrochloride, dyclonine and dyclonine hydrochloride.

In one embodiment, the present invention relates to a conjugate of formula (II) as described above, characterized in that MA is an antiseptic agent selected from the following active ingredients: alcohols, quaternary ammonium compounds, boric acid, chlorhexidine and chlorhexidine derivatives, phenols, terpenes, bactericides, disinfectants, including thimerosal, phenol, thymol, benzalkonium chloride, benzethonium chloride, chlorhexidine, cetylpyridolium chloride and trimethylammonium bromide.

In one embodiment, the present invention relates to a conjugate of formula (II) as described above, characterized in that MA is an anti-inflammatory agent selected from the following active ingredients: non-steroidal anti-inflammatory agents (NSAIDs), propionic acid derivatives, such as ibuprofen and naproxen, acetic acid derivatives, such as indomethacin, enolic acid derivatives, such as meloxicam, acetaminophen, methyl salicylate, monoglycol salicylate, aspirin, mefenamic acid, flufenamic acid, diclofenac, alclofenac, diclofenac sodium, ibuprofen, ketoprofen, naproxen, pranoprofen, fenoprofen, sulindac, fenclofenac, clidanac, flurbiprofen, fentiazac, bufexamac, piroxicam, oxyphenbutazone, pentazocine, tiaramide hydrochloride, steroids, such as clobetasol propionate, betamethasone dipropionate, halbetasol proprionate, diflorasone diacetate, fluocinonide, halcinonide, amcinonide, deoximetasone, triamcinolone acetonide, mometasone furoate, fluticasone, betamethasone dipropionate, triamcinolone acetonide, fluticasone propionate, desonide, fluocinolone acétonide, hydrocortisone vlaerate, prednicarbate, triamcinolone acetonide, fluocinolone acetonide, hydrocortisone and others known from the art, predonisolone, dexamethasone, fluocinolone acetonide, hydrocortisone acetate, predonisolone acetate, methylpredonisolone, dexamethasone acetate, betamethasone, betamethasone fluorine, fluoramethasone and can be one of the less potent corticosteroids, such as hydrocortisone, hydrocortisone-21-monoesters (for example, hydrocortisone-21-acetate, hydrocortisone-21-butyrate, hydrocortisone-21-propionate, hydrocortisone-21-valerate, etc.), hydrocortisone-17,21-diesters (for example, hydrocortisone-17,21-diacetate, hydrocortisone-17-acetate-21-butyrate, hydrocortisone-17,21-dibutyrate, etc.), alclometasone, dexamethasone, flumethasone, prednisolone or methylprednisolone, or can be a more potent corticosteroid, such as clobetasol propionate, betamethasone benzoate, betamethasone dipropionate, diflorasone diacetate, fluocinonide, mometasone furoate, triamcinolone acetonide.

In one embodiment, the present invention relates to a conjugate of formula (II) as described above, characterized in that MA is an analgesic agent selected from the following active ingredients: alfentanil, benzocaine, buprenoiphine, butorphanol, butamben, capsaicin, clonidine, codeine, dibucaine, enkephalin, fentanyl, hydrocodone, hydromorphone, indomethacin, lidocaine, levorphanol, meperidine, methadone, morphine, oxomophine, nicomorphine, oxymorphone, pentazocine, pramoxine, proparacaine, propoxyphene, proxymetacaine, sufentanil, tetracaine and tramadol.

In one embodiment, the present invention relates to a conjugate of formula (II) as described above, characterized in that MA is an anesthetic agent selected from the following active ingredients: phenol, chloroxylenol, dyclonine, ketamine, menthol, pramoxine, resorcinol, procaine-based drugs, such as benzocaine, bupivacaine, chloroprocaine, cinchocaine, cocaine, dexivacaine, diamocaine, dibucaine, etidocaine, hexylcaine, levobupivacaine, lidocaine, mepivacaine, oxethazaine, prilocaine, procaine, proparacaine, propoxycaine, pyrrocaine, risocaine, rodocaine, ropivacaine, tetracaine, and derivatives, such as pharmaceutically acceptable salts and esters, including bupivacaine HCl, chloroprocaine HCl, diamocaine cyclamate, dibucaine HCl, dyclonine HCl, etidocaine HCl, levobupivacaine HCl, lidocaine HCl, mepivacaine HCl, pramoxine HCl, prilocaine HCl, procaine HCl, procaine HCl, propoxycaine HCl, ropivacaine HCl and tetracaine HCl.

In one embodiment, the present invention relates to a conjugate of formula (II) as described above, characterized in that MA is an antihemorrhagic agent selected from the following active ingredients: protamine sulfate, aminocaproic acid, tranexamic acid, carbazochrome, sodium sulfanate, carbazochrome, rutin and hesperidin.

Nanoparticles

Another subject matter of the invention is a nanoparticle comprising a compound of the invention. More specifically, a compound of the invention is present as a constituent, more preferably as a main component of the nanoparticle, which means that the compound of the invention can represent more than 50% by weight, for example, more than 60%, 70%, 80%, 90%, 95%, 98%, 99% or 99.5% by weight of the total weight of the nanoparticle. In some embodiments, the nanoparticle is formed by a compound of the invention. In other words, the nanoparticle results from the self-organization of the molecules of the compound of the invention.

One embodiment of the present invention relates to a nanoparticulate system based on the formation of ion pairs between charged linear terpene molecules (positive or negative) according to formula (I) of the present invention (such as phytol or derivatives) and charged MA active molecules (negative or positive, respectively) without the need for covalent coupling.

It is thus possible to adjust the quantity of charged linear terpene molecules (positive or negative) according to formula (I), according to the present invention relative to the active molecules according to the present invention in order to obtain the nanoparticles. The ratio (i.e. the index “k” in formula II) between molecules of charged linear terpenes (positive or negative) according to formula (I) of the present invention relative to the active molecules MA can vary between 0.1 and 6. Preferably, k ranges between 0.5 and 5.5, between 0, 7 and 5, between 1 and 4, between 1.5 and 3, or even between 2 and 3. Preferably, k is a substantially whole number selected from 1, 2, 3, 4, 5 and 6. The term “substantially” is understood herein to be a variation of plus or minus 0.1.

In addition, the one or more conjugated molecule(s) of active ingredient(s) simply can be covalently linked, directly or via a spacer, to a linear terpene according to the present invention (such as phytol or a phytol derivative).

The covalent coupling of the considered active ingredient with a linear terpene according to the present invention (such as phytol) does not pose any problem to a person skilled in the art. In this way, the present invention makes it possible to exploit the unexpected property of the linear terpene according to the present invention to form nanoparticles with the active ingredient according to formula (II) as defined above.

Preferably, the average diameter of said nanoparticles (whether in the form of salts or in the form of molecules comprising only covalent bonds) is within a range from 10 nm to 800 nm, more preferably from 50 nm to 400 nm, and more preferably from 100 nm to 200 nm. Thus, the average hydrodynamic diameter of the nanoparticle of the invention typically ranges from 10 to 800 nm, preferably from 30 to 500 nm, and in particular from 50 to 400 nm. For example, the nanoparticles can have an average hydrodynamic diameter ranging from 70 nm to 200 nm, for example, ranging from 100 nm to 250 nm. The average hydrodynamic diameter preferably is determined by dynamic light diffusion at 20° C. In other words, within the scope of the present invention, mono-dispersed colloidal suspensions of particles having an average diameter ranging from 10 to 800 nm, in particular from 75 to 500 nm, and more preferably from 100 nm to 200 nm, are produced.

Particle size is an important parameter determining the in vivo future of the nanoparticles after oral administration and, as a general rule, for example, sizes less than 500 nm are considered to facilitate interactions with epithelia.

Preferably, the methods for preparing a compound of formula (I) or (II) are well known. A person skilled in the art can refer to the standard procedures. General protocols for preparing the compounds of the invention are provided in the examples of the present application.

For example, patent application WO2012/076824 discloses methods for synthesizing these nanoparticles. The compounds according to the invention are capable of self-organizing into nanoparticles. For example, nanoprecipitation is a common technique that combines the advantages of one-step preparation, easy scaling, and the use of less toxic solvents compared to other manufacturing methods. Thus, the formation of nanoparticles can increase the biological activity of the compound and improve the delivery of these active molecules to cells. In addition, the compound of the invention in the form of nanoparticles can have improved storage stability compared to its free form. The compounds of the invention according to formulas (I) and (II) can be in the form of nanoparticles or in formulations intended to produce nanoparticles, i.e. intended to be placed in an aqueous solution.

For example, the nanoparticles of the compound of formula (I) and/or (II) can be obtained by dissolving the compound in an organic solvent, such as acetone or ethanol, then adding this mixture to an aqueous phase with stirring leading to the formation of nanoparticles with or without surfactant(s). Surfactants include, for example, polyoxyethylene-polyoxypropylene copolymers, sodium lauryl sulfate, phospholipid derivatives and lipophilic derivatives of polyethylene glycol. The invention also relates to a colloidal system containing the particles of the invention, preferably in an aqueous medium.

More particularly, the nanoparticles according to the present invention can be obtained using a method comprising at least the following steps:
providing a solution of a compound of formula (II) as defined above in a water-soluble organic solvent;
pouring said organic solution into an aqueous phase, which, with stirring, instantly forms the expected nanoparticles in suspension in said aqueous phase; and
where appropriate, isolating said nanoparticles.

Therapeutic Application

The compound of formula (I) or (II), a nanoparticle according to the present invention, as well as any particular compound described herein, can be used as a drug.

The present invention further relates to a compound according to formulas (I) or (II) according to the present invention or to a pharmaceutical composition according to the present invention for its use as a drug intended for treating cancer, allergies, in particular skin allergies, inflammatory reactions, in particular inflammatory reactions of the skin, such as dermatitis, eczema, psoriasis, vitiligo, erythema, inflammatory alopecia, viral infections, bacterial infections, respiratory diseases, such as asthma, skin conditions, such as acne, autoimmune diseases, pain, neurodegenerative diseases, myopathies, osteopathies, hepatitis, renal insufficiency, urogenital diseases, eye diseases, diseases of the digestive tract and/or blood diseases.

The present invention also relates to said composition for its use as a drug for the treatment and/or prevention of the aforementioned diseases and/or conditions.

In another aspect, the invention therefore relates to a pharmaceutical composition comprising a compound of formula (I) or a salt or solvate thereof, a nanoparticle of the invention, as well as any particular compound described herein and a pharmaceutically acceptable excipient. The compound or the nanoparticle of the invention is present as an active ingredient in said pharmaceutical composition.

The pharmaceutical composition of the invention can comprise:
from 0.01 to 90% by weight of a compound or of a nanoparticle of the invention, and from 10% to 99.99% by weight of one or more pharmaceutically acceptable excipient(s), the percentage being expressed relative to the total weight of the composition. Preferably, the pharmaceutical composition can comprise:
from 0.1% to 50% by weight of a compound or of a nanoparticle of the invention, and from 50% to 99.9% by weight of pharmaceutically acceptable excipients.

The invention also relates to a method for treating or preventing a disease in a subject, said method comprising administering the subject a therapeutically effective amount of a compound of formula (I) or of a nanoparticle as defined above.

The term “a therapeutically effective amount or dose” is understood, within the scope of the present invention, to mean an amount of the compound of the invention that prevents, eliminates, slows down the considered disease or reduces or delays one or more symptoms or disorders caused by or associated with said disease in the subject, preferably a human. The effective amount, and more generally the dosage regimen, of the compound of the invention and of its pharmaceutical compositions can be determined and adapted by a person skilled in the art. An effective dose can be determined using conventional techniques and observing the results obtained under like circumstances. The therapeutically effective dose of the compound of the invention will vary depending on the disease to be treated or prevented, its severity, the route of administration, any co-therapy involved, the age of the patient, their weight, their general state of health, medical history, etc. Typically, the amount of the compound to be administered to a patient can range from approximately 0.01 mg/kg to 500 mg/kg of body weight, preferably from 0.1 mg/kg to 300 mg/kg of body weight, for example, from 25 to 300 mg/kg.

The compound or the nanoparticle of the invention can be administered to the subject daily for several consecutive days, for example, for 2 to 10 consecutive days, preferably 3 to 6 consecutive days. This treatment can be repeated every two or three weeks or every one, two or three months. Alternatively, the compound or the nanoparticle of the invention can be administered in a single dose once a week, once every two weeks or once a month. The treatment can be repeated one or more times a year.

Advantageously, the approach that is contemplated according to the ionic form of the invention makes it possible to avoid: i) tedious synthesis; ii) the risk of losing the activity of the drug by chemical modification; and iii) the need to break the covalent bonds between the active compounds and the self-assembly agents in order to release the active compounds.

Alternatively, the approach that is contemplated according to the covalent form of the invention, potentially allows molecules to be obtained that are less sensitive to degradation/non-eliminations.

The pharmaceutical composition comprising the compounds according to the present invention can be administered systemically (for example, orally) or locally (for example, topically).

The compound of the invention (for example, in the form of a pharmaceutical, dermatological or cosmetic composition) can be administered by any conventional route including, but not limited to, oral, buccal, sublingual, rectal, intravenous, intramuscular, subcutaneous, intraosseous, dermal, transdermal, mucous membrane, transmucosal, intraarticular, intracardiac, intracerebral, intraperitoneal, intranasal, pulmonary, intraocular, vaginal or transdermal. In fact, the route of administration of the compound of the invention can vary depending on the disease to be treated and on the organ or tissue of the patient suffering from the disease. In some preferred embodiments, the compound of the invention is administered intravenously or orally. As mentioned above, the subject or the patient preferably is a human.

Given their small size, the nanoparticles of the invention can be administered intravenously in the form of an aqueous suspension and are therefore compatible with vascular microcirculation.

Preferably, the present invention is aimed at nanoparticles as defined above, optionally in the form of a lyophilisate, for the preparation of a pharmaceutical composition particularly applicable to mucous membranes, such as the oropharyngeal mucosa, the oral mucosa, the pulmonary mucosa, the vaginal mucosa, the nasal mucosa, and the gastrointestinal mucosa. In some particular embodiments, the pharmaceutical composition can be a lyophilisate or a lyophilized powder. Said powder can be dissolved or suspended in a suitable vehicle just before being administered to the patient, for example, intravenously or orally.

Thus, the present invention also relates to a lyophilisate comprising at least the nanoparticles as described above. According to a preferred embodiment, this lyophilisate further comprises at least one cryoprotectant, in particular trehalose, glycerol and glucose, and more preferably trehalose.

The present invention is thus aimed at dosages in solid forms intended for oral administration containing at least nanoparticles according to the invention, optionally in the form of a lyophilisate, or preparations intended for reconstituting the nanoparticles. This dosage in solid form advantageously can be a dosage in solid form with delayed release, such as, for example, enteric coated tablets or capsules, the surface coating of which ensures the delayed release.

The claimed nanoparticles also can be suitable for administration other than by the oral route, for example, by the topical route or by the subcutaneous route. Finally, the nanoparticles according to the present invention are particularly advantageous compared to a highly improved skin penetration of the product according to the present formulas (I) or (II) of the invention due to the size and the nature of the nanoparticles (hydrocarbon chain).

The pharmaceutical composition can be of any type. More precisely, but by way of an example, the pharmaceutical formulations compatible with the nanoparticles according to the invention can be: intravenous injections or infusions, saline solutions or purified water solutions, compositions for inhalation, creams, ointments, lotions, gels, capsules, sugar-coated tablets, cachets and syrups incorporating, in particular as a vehicle, water, calcium phosphate, sugars, such as lactose, dextrose or mannitol, talc, stearic acid, starch, sodium bicarbonate and/or gelatin.

In a particular embodiment, the pharmaceutical composition can be a solid oral dosage form, a liquid dosage form, a suspension, for example, for the intravenous route, a dosage form for topical application, such as a cream, an ointment, a gel and the like, a transdermal patch, mucoadhesive patch or tablet, in particular an adhesive bandage or dressing, suppository, aerosol for intranasal or pulmonary administration.

As indicated above, the formulation of the active therapeutic compounds considered according to the present invention, in the form of nanoparticles according to the present invention, constitutes an advantageous alternative compared to the formulations that already exist, in several respects.

The present invention thus relates to a pharmaceutical or dermatological composition, in particular a drug, comprising at least one nanoparticle, optionally in the form of a lyophilisate, as described above, in association with at least one acceptable pharmaceutical vehicle.

The pharmaceutically acceptable excipients that can be used are particularly described in the Handbook of Pharmaceutical Excipients, American Pharmaceutical Association (Pharmaceutical Press; 6th revised edition, 2009). Typically, the pharmaceutical composition of the invention can be obtained by mixing a compound of formula (I) as described above or a nanoparticle thereof with at least one pharmaceutical excipient.

When the nanoparticles are used in dispersion in an aqueous solution, they can be combined with excipients such as sequestering or chelating agents, antioxidants, pH regulators and/or buffering agents.

In particular, solid pH-resistant dosage forms are particularly useful for improving the absolute bioavailability of the nanoparticles of the invention relative to the acidic pH of the stomach.

Examples of suitable excipients include, but are not limited to, solvents, such as water or water/ethanol mixtures, fillers, carriers, diluents, binders, anti-caking agents, plasticizers, disintegrants, lubricants, flavors, buffering agents, stabilizers, colorants, colorants, antioxidants, non-stick agents, softeners, preservatives, surfactants, wax, emulsifiers, wetting agents and slip agents. Examples of diluents include, but are not limited to, microcrystalline cellulose, starch, modified starch, calcium phosphate dibasic dihydrate, calcium sulfate trihydrate, calcium sulfate dihydrate, calcium carbonate, mono- or disaccharides, such as lactose, dextrose, sucrose, mannitol, galactose and sorbitol, xylitol and the combinations thereof.

Examples of binders include, but are not limited to, starches, for example, potato starch, wheat starch, corn starch, gums, such as gum tragacanth, acacia gum and gelatin, hydroxypropylcellulose, hydroxyethylcellulose, hydroxypropylmethylcellulose, polyvinylpyrrolidone, copovidone, polyethylene glycol and the combinations thereof.

Examples of lubricants include, but are not limited to, fatty acids and the derivatives thereof, such as calcium stearate, glyceryl monostearate, glyceryl palmitostearate magnesium stearate, zinc stearate or stearic acid, or polyalkylene glycols, such as PEG. The slip can be selected from colloidal silica, silicon dioxide, talc and the like. Examples of disintegrants include, but are not limited to, crospovidone, croscarmellose salts, such as croscarmellose sodium, starches and the derivatives thereof.

Examples of surfactants include, but are not limited to, simethicone, triethanolamine, polysorbates and the derivatives thereof, such as Tween® 20 or Tween® 40, poloxamers, fatty alcohols, such as lauryl alcohol, cetyl alcohol and alkylsulfate, such as sodium dodecylsulfate (SDS). Examples of emulsifiers include, for example, ethyl alcohol, isopropyl alcohol, ethyl carbonate, ethyl acetate, benzyl alcohol, benzyl benzoate, propylene glycol, 1,3-butylene glycol, dimethylformamide, oils, polyethylene glycol and sorbitan fatty acid esters or the mixtures thereof.

In addition to the active compounds, the liquid dosage forms can contain inert diluents commonly used in the art, such as water or other solvents, solubilizers and emulsifiers, such as, for example, ethyl alcohol, isopropyl alcohol, ethyl carbonate, ethyl acetate, benzyl alcohol, benzyl benzoate, propylene glycol, 1,3-butylene glycol, dimethylformamide, oils, polyethylene glycol and the fatty acid esters of sorbitan or mixtures of these substances, and the like. If desired, the composition can also include adjuvants, such as wetting agents, emulsifying agents, suspending agents, anti-oxidants, buffers, pH modifiers and the like.

Suspensions, in addition to the compound or nanoparticle of the invention, can contain suspending agents, such as ethoxylated isostearyl alcohols, polyoxyethylene sorbitol and sorbitan esters, microcrystalline cellulose, aluminum metahydroxide, bentonite, agar-agar and the like. Vaginal or rectal suppositories can be prepared by mixing the compounds of the present invention with suitable non-irritating excipients or carriers, such as cocoa butter, polyethylene glycol or a suppository wax that is solid at ordinary temperatures but is liquid at body temperature and, consequently, melts into the rectum or vaginal cavity and releases the active component. Ointments, pastes, creams and gels can contain, in addition to an active compound of this invention, excipients such as animal and vegetable fats, oils, waxes, paraffins, starch, tragacanth, cellulose derivatives, polyethylene glycols, silicones, bentonites, silicic acids, talc and zinc oxide, or the mixtures thereof.

It is obvious that the one or more excipient(s) to be combined with the active compound of the invention can vary according to (i) the physicochemical properties, in particular the stability of said active compound, (ii) the desired pharmacokinetic profile for said active compound, (iii) the dosage form, and (iv) the route of administration.

Oral solid dosage forms include, but are not limited to, tablets, capsules, pills, and granules. Optionally, said oral solid forms can be prepared with coatings and shells, such as enteric coatings or other suitable coatings or shells. Several of these coatings and/or shells are well known in the art. Examples of coating compositions that can be used are polymeric substances and waxes. The liquid dosage forms include pharmaceutically acceptable emulsions, solutions, suspensions, syrups and elixirs.

### FIGURES

FIG. 1 is a graph showing the stability of the nanoparticulate suspensions over time for conjugates 3 (dashed line) and conjugates 4 (solid line).

FIG. 2 is a graph showing the stability of the nanoparticulate suspensions over time for conjugates 7 (large spaced dashed line), conjugates 8 (solid line) and conjugates 9 (closely spaced dashed line).

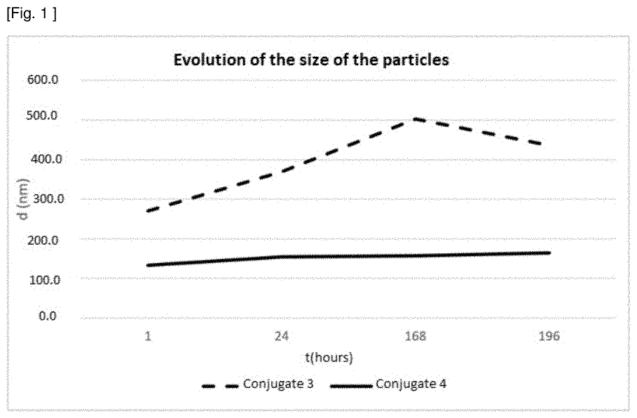

FIG. 1 is a histogram showing the variation of areas as a function of time for conjugate 9 (black column) and retinol (gray column), at 0, 1 and 2 days.

### EXAMPLES

The following abbreviations are provided for the following examples:

CAS: “Chemical Abstracts Service” English international standard.

Dm: Dermis.

Ø: Diameter.

Ep: Epidermis.

e: Thickness.

FZ: Franz type diffusion cell.

h: Hour.

INCI: International Nomenclature of Cosmetic Ingredients.

LOD: Limit of detection threshold.

LOQ: Limit of quantification threshold.

LR: Liquid Receiver.

OECD: Organization for Economic Cooperation and Development.

PBS: Phosphate buffered saline solution (pH 7.4).

IWL: Insensible Water Loss.

SC: Stratum Corneum.

sem: Standard error of the mean.

SD: Standard deviation.

V: Volume.

rpm: revolution(s) per minute.

The ¹H and ¹³C NMR spectra were measured on a Brucker Advance 300 MHz spectrometer in CDCl₃ using tetramethylsilane (TMS) as a reference. Chemical shifts are expressed in ppm.

The mean particle sizes were measured using the “Dynamic Light Scattering” (DLS) method on a Malvern-Panalytical Nano-Sizer ZS® at 25° C. with a detection angle of 173° and a wavelength of 633 nm. The reported sizes are determined by the mean of three measurements. The measurements were carried out in polystyrene cuvettes.

The HPLC analyzes were carried out with an “Ultimate 3000 system” chain, Dionex®, France on a C18 “Vintage series KR C18—5 μm-150×4.6 mm (Interchim®) column. The samples were detected by UV absorption at λ=325 nm.

### Example 1: Synthesized Products

### Preparation of Phytyl Mono-Succinate

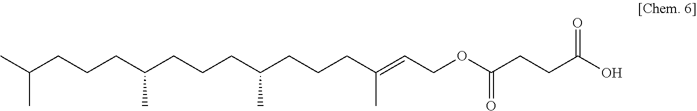 [SMILES: C/C(=C\COC(=O)CCC(=O)O)CCC[C@H](C)CCC[C@H](C)CCCC(C)C]

The following are added to a solution of phytol (10.00 g, 33.78 mmol, 1.0 equiv) and succinic anydride (3.54 g, 35.47 mmol, 1.05 equiv) in PhMe (135 mL): Et₃N (5.40 mL, 38.85 mmol, 1.05 equiv), then DMAP (204 mg, 1.69 mmol, 0.05 equiv), and the reaction is heated to 50° C. with stirring for 7 h.

The TLC analysis (EtOAC/CyH—60:40, revealed by CAM) shows complete conversion of the starting material. The formation of the desired compound is confirmed by a comparison with an authentic sample.

The reaction medium is hydrolyzed with aq. saturated NH₄Cl, then transferred to a separating funnel and the organic phase is separated. The aqueous phase is extracted with EtOAc (3×50 mL). The organic extracts are combined, washed with aq. HCl (0.1 N), dried over MgSO₄, filtered and concentrated under reduced pressure.

The concentrate is then purified by silica gel chromatography (EtOAc/CyH—30:70 to 50:50) to provide the expected compound (12.84 g, 32.42 mmol, 96%) in the form of a yellow oil.

¹H NMR (300 MHz, CDCl₃): δ 5.33 (td, J=7.1, 1.3 Hz, 1H), 4.62 (d, J=7.1 Hz, 2H), 2.91-2.47 (m, 4H), 1.97 (t, J=7.6 Hz, 2H), 1.72 (brs, 3H), 1.55-1.00 (m, 19H), 0.92-0.73 (m, 12H) ppm.

¹³C NMR (75 MHz, CDCl₃): δ 178.3, 172.2, 142.8, 117.9, 61.7, 39.8, 39.4, 37.4, 37.4, 37.3, 36.6, 32.8, 32.7, 29.0, 28.9, 27.8, 25.0, 24.8, 24.5, 22.7, 22.6, 19.7, 19.7, 16.3 ppm.

### Preparation of Phytyl Mono-Dithioglycolate

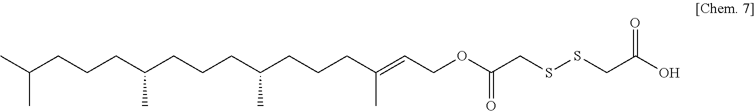 [SMILES: C/C(=C\COC(=O)CSSCC(=O)O)CCC[C@H](C)CCC[C@H](C)CCCC(C)C]

Dithioglycolic acid (0.5 g, 2.74 mmol, 2.95 equiv) and acetic anhydride (2 ml) are stirred under an inert atmosphere for 2 h at 21° C. Then the mixture is azeotropically distilled under reduced pressure with PhMe (3×20 mL), while controlling the temperature of the bath (<30° C.). The residue that is obtained is then used in the next step without further purification. The anhydride that is obtained is dissolved in CH₂Cl₂ (20 mL), then phytol (275 mg, 0.928 mmol, 1.0 equiv) and DMAP (11 mg, 0.092 mmol, 0.1 equiv) are added. The reaction is stirred at 21° C. for 1 h and the end of the reaction is checked by TLC (EtOAc/CyH=1:1). The crude compound is then isolated by filtration and dried under reduced pressure (T<30° C.) to yield a yellow semi-solid (0.627 g). The residue that is obtained is then purified by silica gel chromatography (EtOAc/CyH=20:80+1% AcOH) to yield the expected compound in the form of a yellow solid (338 mg, 0.734 mmol, 79%).

¹H NMR (300 MHz, CDCl₃): δ 10.84 (s, 1H), 5.37 (t, J=7.2 Hz, 1H), 4.69 (dd, J=7.0, 3.7 Hz, 2H), 3.63 (dd, J=11.6, 3.9 Hz, 5H), 2.18-1.90 (m, 2H), 1.72 (d, J=3.6 Hz, 4H), 1.62-0.98 (m, 20H), 0.87 (td, J=6.3, 4.0 Hz, 12H) ppm.

General Procedure A for Molecules Containing Carboxylic Acids:

The following is added to a solution of carboxylic acid (1.05 equiv) in CH₂Cl₂ (0.2 M): EDC-HCl (1.1 equiv) and the reaction medium is stirred for 10 min. Phytol (1.0 equiv) followed by DMAP (0.1 equiv) are added and the reaction medium is stirred at 21° C. for 12 h.

The reaction medium is hydrolyzed with aq. saturated NH₄Cl, then transferred to a separating funnel and the organic phase is separated. The aqueous phase is extracted with EtOAc (3×30 mL). The organic extracts are combined, washed with aq. saturated NaCl (2×30 mL), dried over MgSO₄, filtered and concentrated under reduced pressure.

### Phytyl Nicotinate

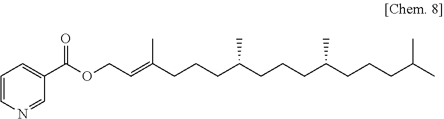 [SMILES: C/C(=C\COC(=O)c1cccnc1)CCC[C@H](C)CCC[C@H](C)CCCC(C)C]

Prepared from nicotinic acid (200 mg, 1.625 mmol).

The residue that is obtained is purified by silica gel chromatography (EtOAc/CyH—0:100 at 20:80) to provide the expected compound (474 mg, 1.182 mmol, 73%) in the form of a yellow oil.

¹H NMR (300 MHz, CDCl₃): δ 9.24 (s, 1H), 8.78 (d, J=3.8 Hz, 1H), 8.36 (dt, J=7.8, 1.9 Hz, 1H), 7.44 (dd, J=7.8, 5.0 Hz, 1H), 5.46 (tq, J=7.2, 1.2 Hz, 1H), 4.88 (d, J=7.2 Hz, 2H), 2.04 (t, J=7.6 Hz, 2H), 1.76 (d, J=1.2 Hz, 3H), 1.57-1.00 (m, 19H), 0.88-0.80 (m, 12H) ppm.

¹³C NMR (75 MHz, CDCl₃): δ 165.0, 153.1, 150.9, 143.2, 136.8, 126.3, 123.0, 117.7, 62.1, 39.8, 39.3, 37.3, 37.3, 37.2, 36.5, 32.7, 32.6, 27.9, 24.9, 24.7, 24.4, 22.6, 22.5, 19.7, 19.6, 16.4 ppm.

### Phytyl Sinapinate

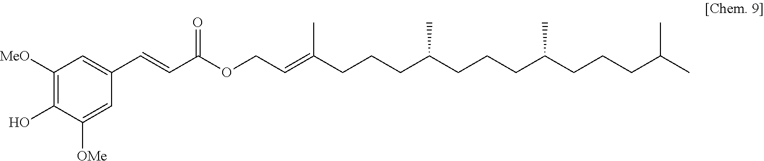 [SMILES: COc1cc(/C=C/C(=O)OC/C=C(\C)CCC[C@H](C)CCC[C@H](C)CCCC(C)C)cc(OC)c1O]

Prepared from sinapic acid (113 mg, 0.530 mmol).

The residue that is obtained is purified by silica gel chromatography (EtOAc/CyH—0:100 to 30:70) to provide the expected compound (205 mg, 0.341 mmol, 67%) in the form of a waxy white solid.

¹H NMR (300 MHz, CDCl₃): δ 7.75 (d, J=15.9 Hz, 1H), 6.78 (s, 2H), 6.39 (d, J=15.9 Hz, 1H), 5.34 (tq, J=7.2, 1.2 Hz, 1H), 4.64 (d, J=7.2 Hz, 2H), 3.85 (s, 6H), 2.98 (t, J=7.2 Hz, 2H), 2.77 (t, J=7.2 Hz, 2H), 2.00 (t, J=7.5 Hz, 2H), 1.69 (s, 3H), 1.58-0.98 (m, 19H), 0.89-0.82 (m, 12H) ppm.

¹³C NMR (75 MHz, CDCl₃): δ 172.1, 172.0, 170.0, 152.5, 146.7, 142.9, 132.4, 130.8, 118.0, 117.7, 105.0, 61.8, 56.2 (2C), 39.9, 39.4, 37.5, 37.4, 37.3, 36.7, 32.8, 32.7, 29.8, 29.4, 28.9, 28.0, 25.1, 24.8, 24.5, 22.8, 22.7, 19.8, 19.8, 16.4 ppm.

### Phytyl Ibuprofenate

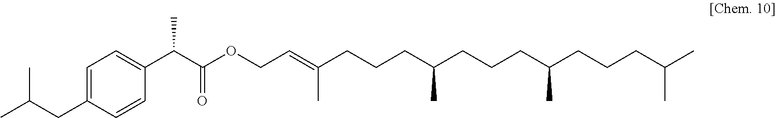 [SMILES: C/C(=C\COC(=O)[C@@H](C)c1ccc(CC(C)C)cc1)CCC[C@H](C)CCC[C@H](C)CCCC(C)C]

Prepared from ibuprofen (206 mg, 1.00 mmol).

The residue that is obtained is purified by silica gel chromatography (EtOAc/CyH—30:70) to provide the expected compound (426 mg, 0.880 mmol, 88%) in the form of a pale yellow oil.

¹H NMR (300 MHz, CDCl₃): δ 7.49-7.35 (d, 2H), 7.28-7.13 (d, 2H), 5.49 (t, 1H), 4.79-4.66 (d, 2H), 3.46 (q, 1H), 2.62-2.45 (d, 2H), 2.25 (t, 2H), 2.04-2.00 (s, 3H), 1.77-1.73 (m, 1H), 1.66 (m, 1H), 1.59 (d, 3H), 1.57-1.22 (m, 14H), 1.07-1.06 (2d, 6H), 1.11 (s, 25H), 1.02-0.93 (m, 12H) ppm.

¹³C NMR (75 MHz, CDCl₃): δ 177.1, 142.0, 140.1, 140.0, 131.1, 131.0, 126.5, 126.5, 121.2, 61.5, 45.7, 45.3, 39.4, 39.3, 36.81 (3C), 35.8, 34.82 (2C), 28.3, 27.6, 25.1, 23.9, 23.7, 22.73 (2C), 22.2 (2C), 20.40 (2C), 20.3, 16.5 ppm.

### Phytyl Diclofenate

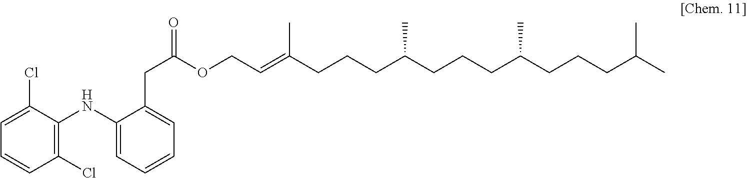 [SMILES: C/C(=C\COC(=O)Cc1ccccc1Nc1c(Cl)cccc1Cl)CCC[C@H](C)CCC[C@H](C)CCCC(C)C]

Prepared from diclofenac (296 mg, 1.00 mmol).

The residue that is obtained is purified by silica gel chromatography (EtOAc/CyH—15:85) to provide the expected compound (473 mg, 0.83 mmol, 83%) in the form of a pale yellow oil.

¹H NMR (300 MHz, CDCl₃): δ 7.36 (d, J=7.5 Hz, 1H), 7.23 (t, J=7.5 Hz, 1H), 7.13 (d, J=7.5 Hz, 2H), 7.03 (d, J=7.5 Hz, 1H), 6.93 (t, J=7.5 Hz, 1H), 6.80 (t, J=7.5 Hz, 1H), 5.48 (t, J=6.2 Hz, 1H), 4.80 (s, 1H), 4.73 (d, J=6.2 Hz, 2H), 3.61 (s, 2H), 2.08 (t, J=5.5 Hz, 2H), 1.66 (t, J=2.9 Hz, 4H), 1.65-1.60 (m, 2H), 1.54 (dq, J=14.6, 7.2 Hz, 1H), 1.43-1.15 (m, 16H), 1.01 (d, J=6.4 Hz, 12H) ppm.

¹³C NMR (75 MHz, CDCl₃): δ 172.4, 145.7, 140.1, 137.9, 132.4, 130.62, 130.6, 130.3, 129.8 (2C), 123.5, 122.2, 121.2, 120.5, 118.7, 60.8, 39.3, 39.3, 36.8 (3C), 36.3, 35.8, 34.8 (2C), 28.3, 25.1, 23.9, 23.7, 22.7 (2C), 20.4, 20.4, 16.5 ppm.

General Procedure B for Molecules Containing Alcohols:

The following is added to a solution of phytyl mono-succinate (1.05 equiv) in CH₂Cl₂ (0.2 M): EDC-HCl (1.1 equiv) and the reaction medium is stirred for 10 min. The corresponding alcohol (1.0 equiv) followed by DMAP (0.1 equiv) are added and the reaction medium is stirred at 21° C. for 12 h.

The reaction medium is hydrolyzed with aq. NH₄Cl, then transferred to a separating funnel and the organic phase is separated. The aqueous phase is extracted with EtOAc (3×30 mL). The organic extracts are combined, washed with aq. saturated NaCl (2×30 mL), dried over MgSO₄, filtered and concentrated under reduced pressure.

### Phytyl (4-tert-butyl cyclohexyl) succinate

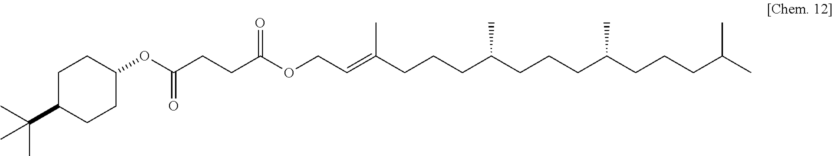 [SMILES: C/C(=C\COC(=O)CCC(=O)O[C@H]1CC[C@H](C(C)(C)C)CC1)CCC[C@H](C)CCC[C@H](C)CCCC(C)C]

Prepared from 4-tert-butyl cyclohexanol (79 mg, 0.530 mmol, as an 80:20 mixture of cis and trans isomers).

The residue that is obtained is purified by silica gel filtration (10 cm) and eluted with EtOAc/CyH (10:90) to provide the expected compound (260 mg, 0.488 mmol, 97%, isolated as an 80:20 mixture of cis and trans isomers) as a colorless oil.

¹H NMR (300 MHz, CDCl₃): δ 5.33 (m, 1H), 4.66 (m, 3H), 2.60 (m, 4H), 1.98 (t, J=7.5 Hz, 4H), 1.80 (m, 2H), 1.66 (brs, 3H), 1.59-0.98 (m, 28H), 0.93-0.75 (m, 21H) ppm.

¹³C NMR (75 MHz, CDCl₃): δ 172.3, 171.8, 142.7, 118.1, 47.2, 39.9, 39.5, 37.5, 37.5, 37.4, 36.7, 32.88, 32.8, 32.3, 32.1, 29.8, 29.6, 29.4, 28.1, 27.7, 27.5, 25.5, 25.1, 24.9, 24.6, 22.8, 22.7, 19.8, 19.8, 16.4 ppm.

### (Vanillyl) Phytyl Succinate

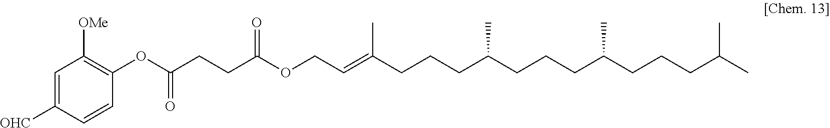 [SMILES: COc1cc(C=O)ccc1OC(=O)CCC(=O)OC/C=C(\C)CCC[C@H](C)CCC[C@H](C)CCCC(C)C]

Prepared from vanillin (900 mg, 1.316 mmol).

The residue that is obtained is purified by silica gel chromatography (EtOAc/CyH—5:95 to 15:85) to provide the expected compound (521 mg, 0.489 mmol, 57%) in the form of a pale yellow oil.

¹H NMR (300 MHz, CDCl₃): δ 9.94 (s, 1H), 7.49 (s, 1H), 7.46 (dd, J=7.8, 1.8 Hz, 1H), 7.23 (d, J=7.8 Hz, 1H), 5.34 (td, J=7.1, 1.1 Hz, 1H), 4.64 (d, J=7.1 Hz, 2H), 3.89 (s, 3H), 2.95 (t, J=6.8 Hz, 2H), 2.76 (t, J=6.9 Hz, 2H), 2.08-1.92 (m, 2H), 1.69 (s, 3H), 1.56-1.01 (m, 19H), 0.84 (dd, J=9.3, 3.7 Hz, 12H) ppm.

¹³C NMR (75 MHz, CDCl₃): δ 190.9, 171.9, 169.9, 151.9, 144.9, 142.9, 135.3, 124.6, 123.4, 117.9, 110.9, 61.8, 56.0, 39.9, 39.4, 37.4, 37.4, 37.3, 36.6, 32.8, 32.7, 29.2, 29.0, 28.0, 25.0, 24.8, 24.5, 22.7, 22.6, 19.8, 19.7, 16.4 ppm.

### Phytyl Retinyl Succinate

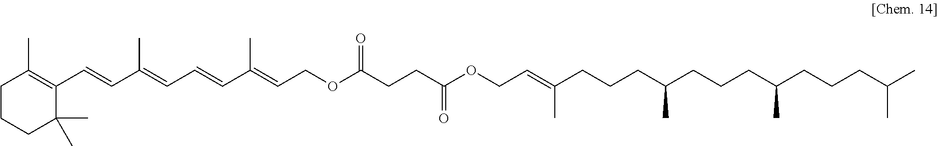 [SMILES: CC1=C(/C=C/C(C)=C/C=C/C(C)=C/COC(=O)CCC(=O)OC/C=C(\C)CCC[C@H](C)CCC[C@H](C)CCCC(C)C)C(C)(C)CCC1]

Prepared from retinol (200 mg, 0.699 mmol).

The residue that is obtained is purified by silica gel chromatography (MTBE/CyH—10:90) to provide the expected compound (115 mg, 0.173 mmol, 25%) in the form of a yellow oil.

¹H NMR (300 MHz, CDCl₃): δ 6.64 (dd, J=15.0, 11.3 Hz, 1H), 6.27 (d, J=15.1 Hz, 1H), 6.18 (d, J=16.2 Hz, 1H), 6.13 (d, J=14.5 Hz, 1H), 6.10 (d, J=16.5 Hz, 1H), 5.60 (t, J=7.1 Hz, 1H), 5.32 (t, J=7.0 Hz, 1H), 4.75 (d, J=7.2 Hz, 2H), 4.61 (d, J=7.1 Hz, 2H), 2.64 (s, 4H), 2.05-1.98 (m, 4H), 1.95 (s, 3H), 1.88 (s, 3H), 1.71 (s, 3H), 1.68 (s, 3H), 1.63-1.05 (m, 23H), 1.02 (s, 6H), 0.85 (t, J=6.3 Hz, 12H).

¹³C NMR (75 MHz, CDCl₃): δ 172.4, 172.3, 143.0, 139.3, 137.9, 137.7, 136.7, 135.9, 130.1, 129.4, 127.1, 125.9, 124.4, 118.0, 61.8, 61.6, 40.0, 39.7, 39.5, 37.5, 37.5, 37.4, 36.8, 34.4, 33.2, 32.9, 32.8, 29.3 (2C), 29.1 (2C), 28.1, 27.1, 25.2, 24.9, 24.6, 22.8, 22.7, 21.8, 19.9, 19.8, 19.4, 16.5, 12.9 ppm.

### (1,3-dimethyl acetonidyl)penthenoyl-(phytyl)-dithiodiglycolate

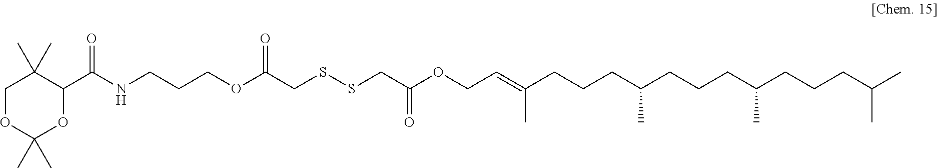 [SMILES: C/C(=C\COC(=O)CSSCC(=O)OCCCNC(=O)C1OC(C)(C)OCC1(C)C)CCC[C@@H](C)CCC[C@@H](C)CCCC(C)C]

Prepared from panthenol acetonide (338 mg, 0.734 mmol).

The residue that is obtained is purified by silica gel chromatography (EtOAc/CyH—20:80) to provide the expected compound (369 mg, 0.536 mmol, 73%) in the form of a colorless oil.

¹H NMR (300 MHz, CDCl₃): δ 6.74 (s, 1H), 5.34 (t, J=6.6 Hz, 1H), 4.66 (d, J=7.2 Hz, 2H), 4.20 (t, J=6.2 Hz, 2H), 4.08 (s, 1H), 3.68 (d, J=11.8 Hz, 1H), 3.51-3.16 (m, 3H), 3.32-3.17 (m, 1H), 1.99 (t, J=7.6 Hz, 2H), 1.95-1.84 (m, 2H), 1.69 (s, 2H), 1.46 (s, 3H), 1.42 (s, 2H), 1.58-0.92 (m, 29H), 1.04 (s, 3H), 0.98 (s, 3H), 0.84 (d, J=6.5 Hz, 8H) ppm.

General Procedure C:

The following are successively added to a solution of carboxylic acid (1.0 equiv) and 2-hydroxyethyl disulfide (5.0 equiv) in THE (0.2 M): DCC (1.3 equiv), DMAP (0.1 equiv), then Et₃N (2 equiv) and the reaction medium is then stirred at 21° C. for 12 h. The reaction medium is then filtered, concentrated under reduced pressure and purified by silica gel chromatography.

Compound “11”:

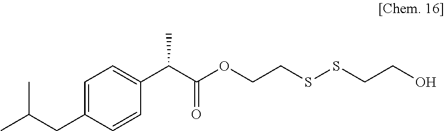 [SMILES: CC(C)Cc1ccc([C@H](C)C(=O)OCCSSCCO)cc1]

Prepared from ibuprofen (206 mg, 1 mmol).

The residue that is obtained is purified by silica gel chromatography (EtOAc/CyH—30:70 to 50:50) to provide the expected compound (250 mg, 0.762 mmol, 76%) in the form of a colorless oil.

¹H NMR (300 MHz, CDCl₃): δ 7.22 (d, J=8.1 Hz, 2H), 7.11 (d, J=8.1 Hz, 2H), 4.44-4.22 (m, 2H), 3.83 (t, J=5.9 Hz, 2H), 3.73 (q, J=7.2 Hz, 1H), 2.88 (t, J=6.7 Hz, 2H), 2.83 (t, J=5.9 Hz, 2H), 2.47 (d, J=7.2 Hz, 2H), 1.97-1.75 (m, 1H), 1.51 (d, J=7.2 Hz, 3H), 0.92 (d, J=6.6 Hz, 6H) ppm.

¹³C NMR (75 MHz, CDCl₃): δ 172.05, 143.14, 133.71, 130.29, 130.29, 130.06, 130.06, 62.69, 61.13, 45.74, 40.95, 40.81, 38.51, 27.63, 22.18, 22.18 ppm.

Compound “12”:

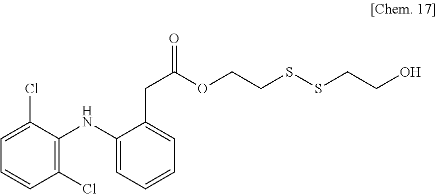 [SMILES: O=C(Cc1ccccc1Nc1c(Cl)cccc1Cl)OCCSSCCO]

Prepared from diclofenac (296 mg, 1 mmol).

The residue that is obtained is purified by silica gel chromatography (EtOAc/CyH—40:60) to provide the expected compound (203 mg, 0.469 mmol, 47%) in the form of a yellow solid.

¹H NMR (300 MHz, CDCl₃): δ 7.35 (d, J=7.5 Hz, 1H), 7.22 (t, J=7.5 Hz, 1H), 7.16 (d, J=7.5 Hz, 2H), 7.01 (d, J=7.5 Hz, 1H), 6.90 (t, J=7.5 Hz, 1H), 6.84 (q, J=7.4 Hz, 1H), 4.47 (s, 1H), 4.44 (t, J=5.0 Hz, 2H), 3.79 (t, J=7.7 Hz, 2H), 3.42 (s, 2H), 2.81 (t, J=5.0 Hz, 2H), 2.74 (t, J=7.7 Hz, 2H) ppm.

¹³C NMR (75 MHz, CDCl₃): δ 172.13, 145.82, 143.69, 132.41, 130.31, 130.18, 130.18, 129.80, 129.80, 123.51, 122.23, 120.13, 118.69, 62.69, 61.13, 40.81, 38.51, 37.01 ppm.

General Procedure D:

The following is added to a cooled solution (0° C.) of diphosgene (2.5 equiv) in CH₂Cl₂ (5 mL): a solution of the corresponding alcohol (1.0 equiv) and DIPEA (5.0 equiv) in CH₂Cl₂ (5 mL). After 45 min stirring at 0° C., the reaction medium is concentrated. The residue is then re-dissolved in CH₂Cl₂ (5 mL), and a solution composed of phytol (1.2 equiv), Et₃N (1.2 equiv) and DMAP (0.1 equiv) in CH₂Cl₂ (5 mL) is then added at 0° C. After 1.5 h with stirring, the reaction medium is hydrolyzed with aq. HCl (1 N, 10 mL), then extracted with CH₂Cl₂ (3×20 mL). The organic phases are combined, washed with aq. saturated NaCl (2×20 mL), then dried over Na₂SO₄, filtered and concentrated under reduced pressure.

Compound “5”:

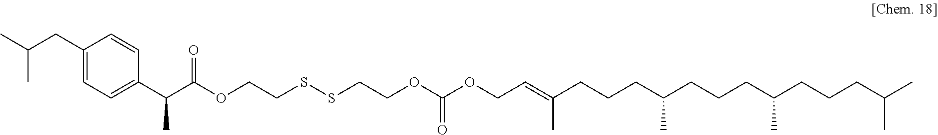 [SMILES: C/C(=C\COC(=O)OCCSSCCOC(=O)[C@@H](C)c1ccc(CC(C)C)cc1)CCC[C@@H](C)CCC[C@@H](C)CCCC(C)C]

Prepared from ibuprofen 11 derivative (420 mg, 1.28 mmol).

The residue that is obtained is purified by silica gel chromatography (EtOAc/CyH—3:97) to provide the expected compound (570 mg, 0.857 mmol, 67%) in the form of a colorless oil.

M=665.05 g/mole

SM: 665.6 [M+H]

Compound “6”:

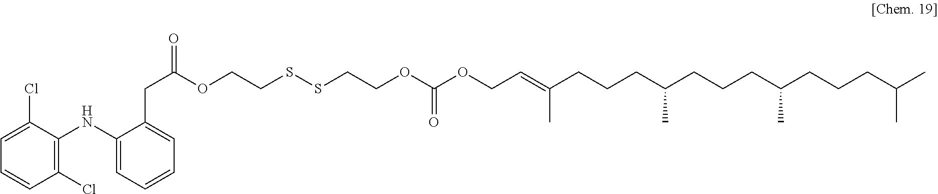 [SMILES: C/C(=C\COC(=O)OCCSSCCOC(=O)Cc1ccccc1Nc1c(Cl)cccc1Cl)CCC[C@@H](C)CCC[C@@H](C)CCCC(C)C]

Prepared from diclofenac derivative 12 (127 mg, 0.295 mmol).

The residue that is obtained is purified by silica gel chromatography (EtOAc/CyH—3:97) to provide the expected compound (138 mg, 0.182 mmol, 62%) in the form of a yellow oil.

M=754.91 g/mole

SM: 754.5 [M+H]

TABLE 1
Examples of synthesized products

| Products | Structures |
|---|---|
| 1 | 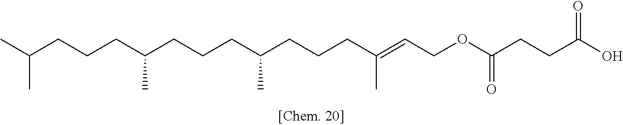 [SMILES: C/C(=C\COC(=O)CCC(=O)O)CCC[C@H](C)CCC[C@H](C)CCCC(C)C] |
| 2 | 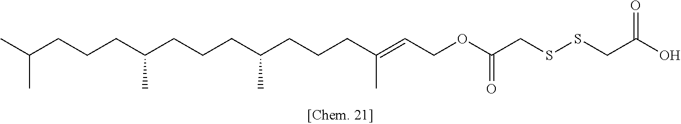 [SMILES: C/C(=C\COC(=O)CSSCC(=O)O)CCC[C@H](C)CCC[C@H](C)CCCC(C)C] |
| 3 | 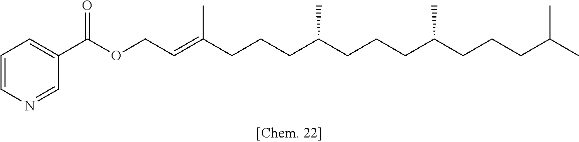 [SMILES: C/C(=C\COC(=O)c1cccnc1)CCC[C@H](C)CCC[C@H](C)CCCC(C)C] |
| 4 | 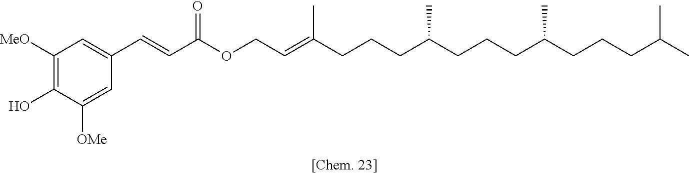 [SMILES: COc1cc(/C=C/C(=O)OC/C=C(\C)CCC[C@H](C)CCC[C@H](C)CCCC(C)C)cc(OC)c1O] |
| 5 | 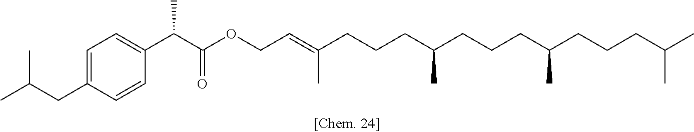 [SMILES: C/C(=C\COC(=O)[C@@H](C)c1ccc(CC(C)C)cc1)CCC[C@H](C)CCC[C@H](C)CCCC(C)C] |
| 6 | 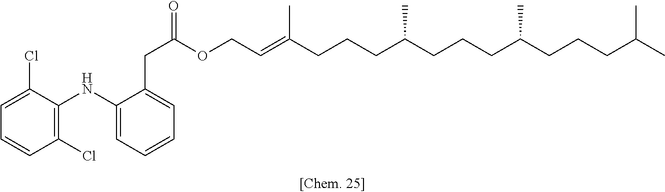 [SMILES: C/C(=C\COC(=O)Cc1ccccc1Nc1c(Cl)cccc1Cl)CCC[C@H](C)CCC[C@H](C)CCCC(C)C] |
| 7 | 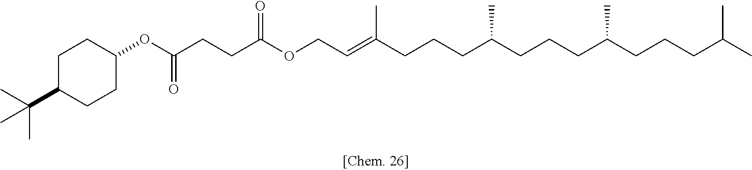 |
| 8 | 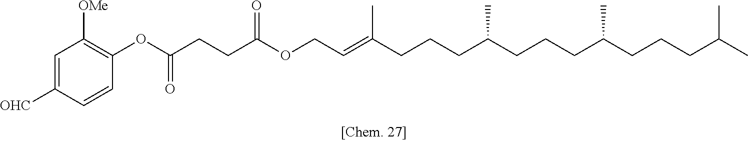 [SMILES: COc1cc(C=O)ccc1OC(=O)CCC(=O)OC/C=C(\C)CCC[C@H](C)CCC[C@H](C)CCCC(C)C] |
| 9 | 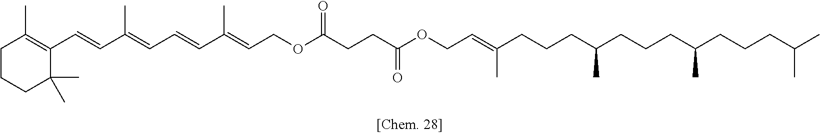 [SMILES: CC1=C(/C=C/C(C)=C/C=C/C(C)=C/COC(=O)CCC(=O)OC/C=C(\C)CCC[C@H](C)CCC[C@H](C)CCCC(C)C)C(C)(C)CCC1] |
| 10 | 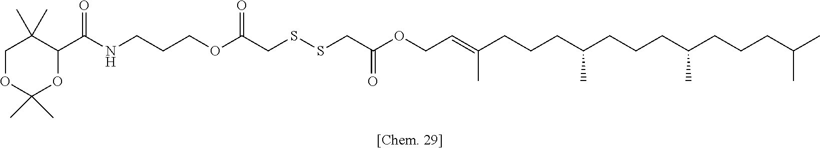 [SMILES: C/C(=C\COC(=O)CSSCC(=O)OCCCNC(=O)C1OC(C)(C)OCC1(C)C)CCC[C@@H](C)CCC[C@@H](C)CCCC(C)C] |
| 11 | 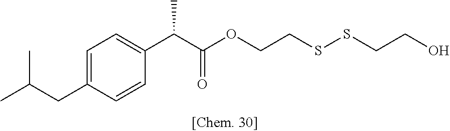 [SMILES: CC(C)Cc1ccc([C@H](C)C(=O)OCCSSCCO)cc1] |
| 12 | 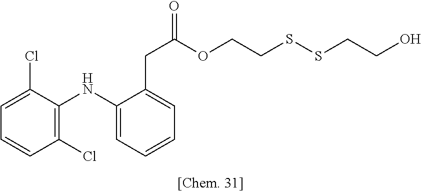 [SMILES: O=C(Cc1ccccc1Nc1c(Cl)cccc1Cl)OCCSSCCO] |
| 13 | 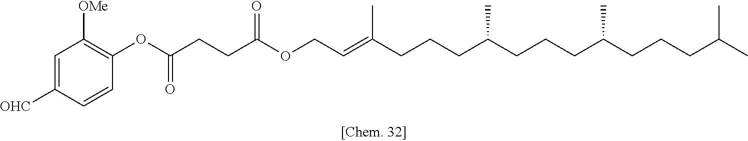 [SMILES: COc1cc(C=O)ccc1OC(=O)CCC(=O)OC/C=C(\C)CCC[C@H](C)CCC[C@H](C)CCCC(C)C] |
| 14 | 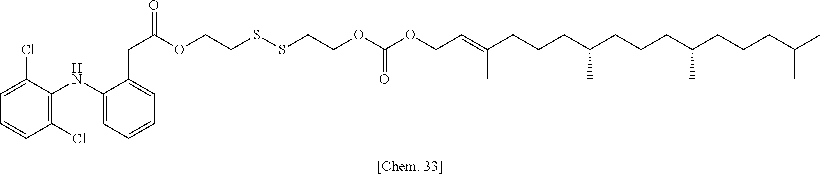 [SMILES: C/C(=C\COC(=O)OCCSSCCOC(=O)Cc1ccccc1Nc1c(Cl)cccc1Cl)CCC[C@@H](C)CCC[C@@H](C)CCCC(C)C] |

With the exception of compounds 11 and 12, these structures all comprise a phytol fragment.

### Example 2: Examples of Self-Assembly

The self-assembly properties were confirmed for all these bio-conjugates. Indeed, nanoobjects could be formed using the nanoprecipitation/solvent evaporation method.

The formation stages are:

(1) Dissolution of the phytolized conjugate in an organic solvent miscible with water;
(2) Nanoprecipitation in water;
(3) Evaporation of the solvent under reduced pressure.

For all the conjugates, the prepared nanoobjects were characterized and generally have the following characteristics:

Observed size ranging between 150 and 170 nm;

Polydispersity index (PDI) ranging between 0.070 and 0.270;

Zeta potential ranging between −19.0 and −35 mV.

The stability of these suspensions was studied over time and shows that the suspensions are stable. In certain borderline cases, the nanoobjects tend to aggregate and lead to precipitation of the conjugates in the medium. However, it has been shown that the stability of these conjugate suspensions can be improved by adding surfactants such as, for example, Pluronic F68. Thus, certain conjugates have had a tendency to aggregate and have been able to lead to stable suspensions for up to 10 days by the addition of 0.5 to 5% (m/m) of Pluronic F68. Other surfactants, like coconut amidopropyl betaine, sodium laureth sulfate, sorbitan palmitate, lauryl glucoside, fatty alcohols, acids and the mixture thereof, phospholipids, phosphatidyl choline have also been used and are currently being studied for their stabilizing effect on conjugate suspensions.

### Example 3: Physico-Chemical Study

Nanoprecipitation:

A solution of conjugate in EtOH (2 mg for 0.5 mL) is added dropwise to vigorously stirred MiliQ water (1 mL). The formation of nanoparticles is observed in that the solution becomes partially cloudy. The suspension that is thus obtained is transferred to a flask and the EtOH is evaporated on a rotary evaporator (200 mbar for 5 min, then 130 mbar for 1 min at 40° C. and at 50 revolutions per minute).

The residual suspension is transferred to a vial and stored at 23° C.

The samples are prepared as follows: 40 μL of the residual suspension is dissolved in 500 μL of MiliQ H₂O.

1. Stability of the Nanoparticulate Suspensions

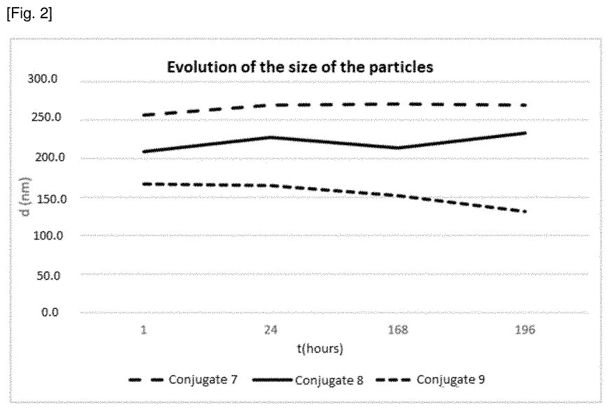

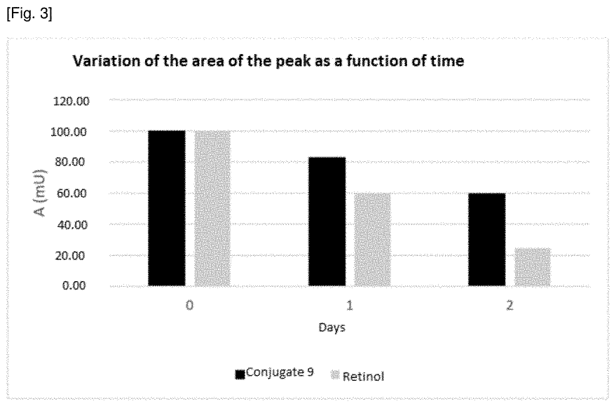

The stability of the suspensions was measured over time and the results are summarized in Tables 2 and 3 and in the graph of FIG. 1 and in the graph of FIG. 2.

Table 2: Stability of the nanoparticulate suspensions over time (Conjugate 3 and 4)

TABLE 2

d (nm)

| Time (h) | Conjugate 3 | Conjugate 4 |
|---|---|---|
| 1 | 132.9 | 270.0 |
| 24 | 154.5 | 369.7 |
| 168 | 156.6 | 502.4 |
| 196 | 164.1 | 436.8 |

Table 3: Stability of the nanoparticulate suspensions over time (Conjugate 7, 8 and 9 and 4).

TABLE 3

| | d (nm) | | |
|---|---|---|---|
| Time (h) | Conjugate 7 | Conjugate 8 | Conjugate 9 |
| 1 | 256.2 | 208.9 | 167.4 |
| 24 | 269.7 | 227.8 | 165.2 |
| 168 | 271.2 | 214.0 | 151.8 |
| 196 | 269.6 | 233.3 | 131.3 |

2. Influence of the Phytolization Process on the Stability of Retinol:

Vitamin A (retinol) is known to be sensitive to oxygen and UV radiation. The phytolization process allows retinol to be stabilized. This protection was demonstrated by HPLC monitoring of a nanoparticulate suspension of the conjugate 9 at 20° C. compared to a solution of retinol under the same conditions.

The solutions of retinol and conjugate 9 in nanoparticulate form (6 mg/L in an H₂O/iPrOH 1:1 mixture) were stored at 21° C. in ambient light and analyzed by HPLC over time (24 and 48 h).

Table 4: HPLC condition.

TABLE 4

| Column | Interchim Vintage series KR C18-5 | |
|---|---|---|
| | micometers -150 × 4.6 mm | |
| T Column | 25° | C. |
| Elution | iPrOH/H2O 85:15 isocratic | |
| Flow | 1 | mL/min |
| Injection volume | 20 | microliters |
| Tr Retinol = | 3.41 | min |
| Tr conjugate 9 = | 11.4 | min |

The variation in the area of the compounds over time is shown over time (average of two measurements)—see Table 5 and the graph of FIG. 3.

Table 5: Variation of areas as a function of time.

TABLE 5

| | t0 | | t24 h | | t48 h | |
|---|---|---|---|---|---|---|
| | Conju- | | Conju- | | Conju- | |
| | gate 9 | Retinol | gate 9 | Retinol | gate 9 | Retinol |
| | A (mU) | A (mU) | A (mU) | A (mU) | A (mU) | A (mU) |
| | 4.1604 | 0.0502 | 3.4609 | 0.0354 | 2.9536 | 0.0218 |
| | 4.0495 | 0.0694 | 3.3380 | 0.0360 | 2.9385 | 0.0273 |
| Average | 4.1050 | 0.0598 | 3.3995 | 0.0357 | 2.9461 | 0.0246 |
| Variation | 0.0000 | 0.0000 | 0.1719 | 0.4030 | 0.2823 | 0.5895 |

CCL: Retinol breaks down twice as quickly when in it is free form.

3. Inclusion of the Conjugates in Cosmetic Formulations:

Preparation of a solution of conjugate 9 at 1%.

800 mg of conjugate 9 is dissolved in EtOH (40 mL), then poured dropwise into H₂O (80 mL) with vigorous stirring (addition 1 mL/min). The suspension is then concentrated in a rotavapor (T=40° C., 50 rpm at 200, then 130 mbar). The volume is then adjusted to 80 mL by adding H₂O.

Face cream:

The face cream was prepared as follows:

Operating mode: Homogenize a phase A (see Table 6), then introduce a phase B (see Table 6) and homogenize for 10 minutes with vigorous stirring (1500 revolutions/min). Produce the emulsion by pouring phase C (see Table 6) into the mixture and then homogenize with vigorous stirring for 10 min. Finally, introduce a phase D (see Table 6).

Table 6: Composition of a face cream.

TABLE 6

| Phase | Ingredients (INCI) | % |
|---|---|---|
| A | Solution of Conjugate 9 at 1% | 75.22 |
| | Glycerin | 3 |
| B | Sodium Polyacrylate | 0.8 |
| C | Coco-Caprylate/Caprate | 6.66 |
| | Caprylic/Capric Triglyceride | 6.66 |
| | Olus Oil | 6.66 |
| D | Phenoxyethanol (and) Ethylhexylglycerin | 1 |

A smooth, pale yellow cream is thus obtained.

Aqueous gel:

A conjugate 9 was included in a cosmetic gel as follows:

Operating mode: Homogenize a phase A (see Table 7) with vigorous stirring (1500 revolutions/min) for 20 minutes. Then, introduce a phase B (see Table 7) and homogenize until the powders have completely dissolved. Prepare the premix for phase C (see Table 7), then introduce it into the mixture and homogenize with vigorous stirring for 15 minutes. Introduce a phase D (see Table 7), then homogenize until the powders have completely dissolved. Finally, adjust to pH 5.0-5.5 with phase E.

Table 6: Composition of an aqueous gel.

TABLE 7

| Phase | Ingredients (INCI) | % |
|---|---|---|
| A | Solution of Conjugate 9 at 1% | 80.2 |
| | Triethyl Citrate | 5 |
| B | Erythritol | 5 |
| C | Xanthan Gum | 1 |
| | Propylene glycol | 3 |
| | Potassium Lactate | 5 |
| D | Aqua (and) Citric Acid | QSP |

A bright yellow gel is thus obtained.

### Example 4: Biological Application

1. Aim

The aim of this study is the ex vivo evaluation, on skin explants of human origin, of the promoting effect of the transdermal passage of an innovative skin vectorization system according to the present invention. The tracer that is used to compare the 2 formulations is retinol.

Each formulation is applied to 3 explants from a single donor. At the end of the contact period (24 hours), the total concentration of retinol is measured in different skin layers (Stratum Corneum, Epidermis and Dermis) and diffusion kinetics are performed on 4 points.

In order to study the promoting effect of the concept of phytolization, two formulas were compared:
F1: Retinol (0.9% retinol equivalent) vectorized with phytol in nanoparticulate form;
F2: Retinol (0.9% retinol equivalent) in free form with a propenetrating agent (5% transcutol) included in the dosage form.

NB: The use of trancutol is regulated and limited to 2.6% for unrinsed body applications.

2. Materials and Methods

2.1 Products Tested and Molecule Assayed

2.1.1. Inclusion of Retinol in Cosmetic Formulations:

Preparation of a solution of conjugate 9 at 1%.

800 mg of conjugate 9 is dissolved in EtOH (40 mL), then poured dropwise into H₂O (80 mL) with vigorous stirring (addition 1 mL/min). The suspension is then concentrated in a rotavapor (T=40° C., 50 rpm at 200, then 130 mbar). The volume is then adjusted to 80 mL by adding H₂O.

Formula F1:

Preparation of a solution of conjugate 9 at 3%.

2.1 g of conjugate 9 is dissolved in EtOH (35 mL), then poured dropwise into H₂O (70 mL) with vigorous stirring (addition 1 mL/min). The suspension is then concentrated in a rotavapor (T=40° C., 50 rpm at 200, then 130 mbar). The volume is then adjusted to 70 mL by adding H₂O.

Formula F1 was prepared as below.

Table 8: formula F1.

TABLE 8

| Phase | Ingredients (INCI) | % |
|---|---|---|
| A | Solution of Conjugate 9 at 3% | 70 |
| | Water | 7.22 |
| | Glycerin | 3 |
| B | Sodium Polyacrylate | 0.8 |
| C | Coco-Caprylate/Caprate | 6.66 |
| | Caprylic/Capric Triglyceride | 6.66 |
| | Olus Oil | 4.66 |
| D | Phenoxyethanol (and) Ethylhexylglycerin | 1 |

Operating mode: Homogenize phase A, then introduce phase B and homogenize for 10 minutes with vigorous stirring (1500 revolutions/min). Make the emulsion by pouring phase C into the mixture, then homogenize with vigorous stirring for 10 min. Finally, introduce phase D.

A smooth, pale yellow cream is thus obtained.

Formula F2:

Preparation of a 1.29% retinol solution containing 7.14% of 2-(2-ethoxyethoxy) ethanol (Transcutol).

0.9 g of retinol is dissolved in EtOH (35 mL), then poured dropwise into an aqueous solution of 2-(2-ethoxyethoxy) ethanol (Transcutol) (5 g in 70 mL) with vigorous stirring (addition 1 mL/min). The suspension is then concentrated in a rotavapor (T=40° C., 50 rpm at 200, then 130 mbar). The volume is then adjusted to 70 mL by adding H₂O.

Formula F2 was prepared as below.

Table 9: formula F2.

TABLE 9

| Phase | Ingredients (INCI) | % |
|---|---|---|
| A | H₂O Solution/Retinol transcultol at 1.28% | 70 |
| | Water | 7.22 |
| | Glycerin | 3 |
| B | Sodium Polyacrylate | 0.8 |
| C | Coco-Caprylate/Caprate | 6.66 |
| | Caprylic/Capric Triglyceride | 6.66 |
| | Olus Oil | 4.66 |
| D | Phenoxyethanol (and) Ethylhexylglycerin | 1 |

Operating mode: Homogenize phase A, then introduce phase B and homogenize for 10 minutes with vigorous stirring (1500 revolutions/min). Make the emulsion by pouring phase PGP-62T 1 C into the mixture, then homogenize with vigorous stirring for 10 min. Finally, introduce phase D.

A smooth, pale yellow cream is thus obtained.

2.2. Materials and Equipment

2.2.1. Biological Material

Human skin samples are obtained from a plastic surgery department of a clinic in Tours (France), after an abdominoplasty operation. Following the operation, the skin is placed in a chamber at a temperature of 4′C, then transferred to our establishment.

Upon receipt, the hypodermis is gently removed and the skin sample is recorded with an encrypted identification number and then stored at −20° C. According to the OECD guidelines (Test N⁰428), the skin can be preserved at this temperature for a maximum period of one year without modifying its permeability.

For this study, 10 skin explants from a single donor are used.

2.3. Sequence of the Study

2.3.1. Characterization of the Explants

A sample of human skin is divided into 10 skin explants measuring 3×3 cm. The explants are thawed at room temperature for 10 minutes, then cleaned with PBS.

The integrity of the skin barrier of each explant is controlled by measuring the insensible water loss (IWL). With the values of IWL measured for the 10 skin explants ranging between 5.2 and 7.6 gm⁻²·h⁻¹, the skin explants are considered suitable for experimentation. The thickness of each explant is measured at five different places.

The average values obtained per formula for these two parameters (IWL and thickness) are shown in Table 10.

Table 10: Average thickness and IWL of skin explants per condition (n=3; *n=1; average±wk).

TABLE 10

| Condition | Thickness (micrometers) | IWL (gm⁻² · h⁻¹) |
|---|---|---|
| F1 | 964 ± 67 | 6.6 ± 1.1 |
| F2 | 856 ± 62 | 6.3 ± 1.1 |
| White* | 881 ± 29 | 5.6 ± 1. |

2.3.2. Transdermal Passage Test

All the skin explants are placed in Franz type diffusion cells with the stratum corneum placed facing the donor compartment. Clips are used to keep the two compartments together and thus provide the seal.

The receiving compartments are filled with receiving liquid. Special care is taken to avoid the formation of air bubbles under the skin explants.

For 1 hour, the diffusion cells are placed on a magnetic plate to keep stirring the receiving liquid, and the whole is placed in an oven enabling a temperature of 32° C. on the surface of the skin and a 50% moisture content to be obtained. The stirring speed of the receiving liquid during the experiment is set to 400 rpm.

After one hour, with the thermal equilibrium of each diffusion cell being reached, the formulations are carefully applied to the surface of the skin explants in accordance with the distribution described in Table 11.

The formulations are in the form of an emulsion, therefore the applications are carried out with a positive displacement pipette.

The amount deposited on the surface of the skin is 500 mg.

The diffusion cells are placed in an oven for 24 hours.

Table 11: Breakdown of explants per condition

TABLE 11

| Condition | Explants |
|---|---|
| White | # T |
| Formula 1 | # 1; # 2; # 3 |
| Formula 2 | # 7; # 8; # 9 |

During the 24 hours of diffusion, 3 kinetic points are achieved at the following times: 1 h, 4 h and 8 h. To this end, a volume of 300 μL of receiving liquid is taken from each cell, then replaced by “new” receiving liquid. Each sample is kept frozen.

At the end of the diffusion time (24 hours), the following procedure is carried out for all the diffusion cells:

Cleaning the skin surface:
Absorption of the unabsorbed fraction;
Cleaning the skin surface with 2 cotton swabs impregnated with micellar water;
Rinsing the skin surface with 2 cotton swabs impregnated with demineralized water;
Drying the skin surface with 1 cotton swab;
Application of D-Squame adhesive to remove product residue remaining on the skin.

Recovery of the Receiving Liquid:
All the receiving liquid is placed in a 15 mL Falcon tube, then frozen.

Recovery of the Stratum Corneum:
Successive application of 2 D-Squame adhesives on the treated area. The two adhesives are placed together in a 15 mL Falcon tube, then frozen, with each adhesive being folded back on itself.

Recovery of the Epidermis and Dermis:
The epidermis and dermis are separated by lightly scraping the surface or, if necessary, by heating at 65° C. for 15 seconds.
The epidermis and dermis are individually placed in a 15 mL Falcon tube, weighed and finally frozen.

2.4. Analysis and Dosage of the Samples

All the samples were recovered.

The extraction and analytical assaying of retinol in the samples was carried out according to the following procedure:

2.4.1. Retinol Assay Method: HPLC.

HPLC analysis procedure:
For the receiving liquids: direct injection
For SC, Ep, Dm: extraction by ethanol (with stirring) before injection.
   Tested extraction time=12 h and 24 h
   Ethanol volume=10 mL for SC, 1 mL for Ep and 2 mL for Dm
   The triplicates of SC, Ep and Dm are “pooled” before extraction
   After extraction, the tubes are centrifuged at 3000 g for 5 minutes
   300 μL is taken for HPLC analyzes

HPCL analysis conditions:
Column: (C18 Vintage series KR C18—5 μm-150×4.6 mm)
Mobile phase: Isopropanol-water (85/15)
Column temperature: 25° C.
Injection volume: 20 μL
Pump flow: 1 mL/min
Detection: UV—325 nm
Retinol retention time: 11.8 min
Total time per injection: 15 mins

3. Transdermal Passage of Retinol from the 2 Formulations

The results of the assaying of retinol in the different skin layers and in the receiving liquids are presented in the following chapters.

3.1. Distribution of Retinol in the Skin Layers

The average amounts of retinol obtained in the skin layers are presented in Table 12 and Table 13.

Table 12: Average amount of retinol (μg/cm²) in the skin layers (12 hours of extraction).

TABLE 12

| | F1 | F3 | Control |
|---|---|---|---|
| Stratum | 10.76 ± 0.25 | 9.34 ± 0.08 | 0.22 |
| Corneum | | | |
| Epidermis | 12.13 ± 0.58 | 6.28 ± 0.10 | 0.22 ± 0.01 |
| Dermis | 0.24 ± 0.06 | 0.22 ± 0.04 | 0.20 ± 0.04 |

Table 13: Average amount of retinol (μg/cm²) in the skin layers (24 hours of extraction).

TABLE 13

| | F1 | F3 | Control |
|---|---|---|---|
| Stratum | 10.67 ± 0.27 | 9.27 ± 0.08 | 0.24 ± 0.03 |
| Corneum | | | |
| Epidermis | 12.46 ± 0.29 | 7.26 ± 0.22 | 0.21 ± 0.03 |
| Dermis | 0.21 ± 0.05 | 0.19 ± 0.02 | 0.17 ± 0.05 |

Two durations for extracting retinol from skin layers were applied: 12 h and 24 h. The results of the test for the transdermal passage of retinol in the skin layers do not show any difference between the values obtained after 12 h of extraction (Table 12) and those obtained after 24 h of extraction (Table 13). This result validates the extraction method.

In the remainder of the document, only the results obtained within 12 hours of extraction are retained and reviewed.

The retinol assay results for the control sample condition show very low values (less than 0.30 μg/cm²) in the 3 layers. This result confirms that the human skin explant used in this study does not contain endogenous retinol.

For the three formulations, the amount of retinol measured in the dermis is of the same order as that of the control sample (˜0.2 μg/cm²). The three formulas do not seem to allow retinol to diffuse into the dermis.

The lowest retinol transdermal diffusion results are obtained with the F2 formulation. In fact, with this formulation, the values obtained in the stratum corneum and in the epidermis are less than 10 μg/cm².

The results of transdermal diffusion of retinol obtained with F1 are higher overall than those obtained with formulation F2. The amount of retinol in the stratum corneum is only slightly higher, with 10.76 μg/cm² for F1 against 9.34 μg/cm² for F2. This is twice as high in the epidermis with 12.13 μg/cm² for F1 against 6.28 μg/cm² for F2, showing the effectiveness of this formulation for transporting the retinol in this skin layer.

3.2. Result of Diffusion of Retinol in the Receiving Fluids

No detection of the molecule of interest was observed in the receiving fluids. This result is consistent with the previous result where there was a very small amount of retinol in the dermis, irrespective of the condition.

4. Conclusions

In conclusion, this study highlights the following elements:
The transdermal absorption of retinol varies depending on the formulation applied to the skin.
Irrespective of the formulation that is used, retinol was not found in the receiving fluid and in the dermis.
Among the 2 formulations that were studied, formulation F2 is the formulation that is the least effective in allowing transdermal diffusion of retinol.

Formulation F1 has the most interesting results in terms of the amount of retinol transported to the stratum corneum and the epidermis, irrespective of the condition.

1. A method of producing conjugates with self-assembly properties comprising a linear terpene, optionally branched, having at most one C═C unsaturation.

2. The method according to claim 1, wherein the terpene comprises between 15 and 25 carbon atoms.

3. The method according to claim 1, wherein the terpene is bio-sourced.

4. The method according to any one of claim 1, wherein the terpene is phytol or a phytol derivative, such as isophytol.

5. A self-assembly agent of formula (I):
X(-Spacer-Y-Terpene)ₚ (I)
wherein:
“Terpene” is as defined in claim 1;
“Y” is a bond or a molecular fragment with a biodegradable bond;
“Spacer” is a bond or a fragment comprising at least one carbon atom;
“X” is a molecular fragment comprising at least one biodegradable bond;
“p” is comprised between 0.1 and 4; and
the “-Spacer-Y-” group optionally can be a bond.

6. The self-assembly agent according to claim 5, wherein the spacer comprises any one of the following fragments: 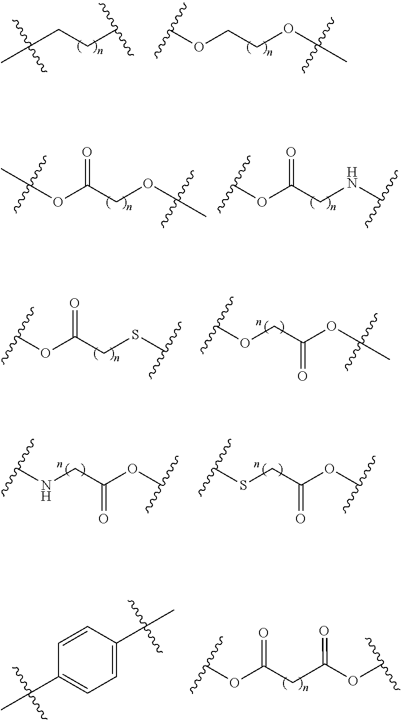
wherein “n” are independently whole numbers ranging between 0 and 6, preferably ranging between 1 and 4.

7. The self-assembly agent according to claim 5, wherein “Y” and/or “X” comprise any one of the following fragments: 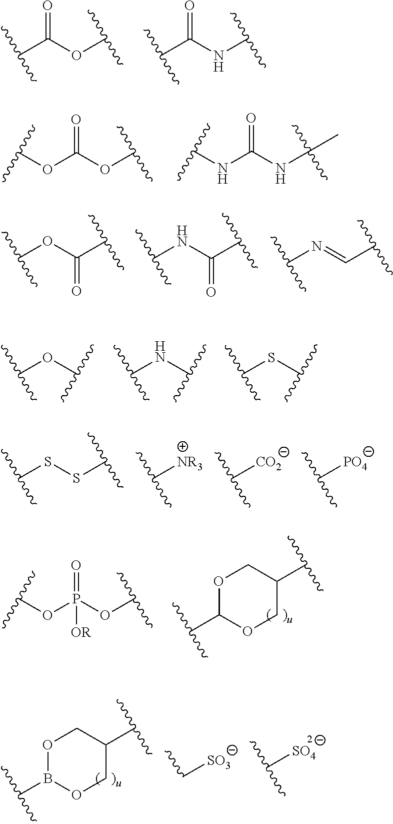
wherein:
“u” are independently whole numbers comprised between 0 and 6, preferably between 0 and 1;
“R” is a hydrogen atom, a C1-C6 alkyl group, a C4-C8 aromatic group or a monocyclic or polycyclic (C1-C6)-aryl (C4-C8) alkyl group, for example, R can represent a hydrogen atom, a methyl, ethyl, propyl, butyl, phenyl, or even a benzyl group.

8. The self-assembly agent according to claim 5, wherein said at least one biodegradable bond of “X” comprises an ionic bond and/or the biodegradable bond of “Y” is a covalent bond.

9. A conjugate with self-assembly properties of formula (II):
MA(-AA)ₖ (II)
wherein
“AA” is a self-assembly agent as defined in claim 5;
“MA” is a biologically active molecule; and
“k” is comprised between 0.1 and 6,
as well as to the pharmaceutically acceptable salts and/or solvates thereof.

10. The conjugate according to claim 9, wherein the MA is selected from ibuprofen, paracetamol, 4-nBu-resorcinol, 6-nHex-resorcinol, azelaic acid, caffeic acid, ferulic acid, glycyrrhizic acid, hyaluronic acid, kojic acid, linoleic acid, lipoic acid, adenosine di-phosphate, adenosine mono-phosphate, adenosine tri-phosphate, aescin, arbutin, bakuchiol, bis-(Et)-Hexyl-dihydroxymethoxybenzyl-malonate, bisabolole, boldine, caffeine, canabidiole, carotenoids, coenzyme A, coenzyme Q10, dihydroxy acetone, dihydroxymethylchromonyl-palmitate, D-panthenol, ectoin, glabridin, idebenone, L-carnitine, licochalchone A, menthol, N-acetyl-tetrapeptide-2, N-acetyl-tetrapeptide-9, niaccinamide, oleuropein, phycocyanin, pro-xylan, resorcinol, resveratrol, superoxide dismutase, tripeptide-29, tyamine pyrrophosphate, vanillin, vitamin A, vitamin B3, vitamin B8, vitamin C and vitamin E.
